(19) United States
(12) Patent Application Publication
(10) Pub. No.: US 2014/0114315 A1
(43) Pub. Date: Apr. 24, 2014

(54) TISSUE MODIFICATION DEVICES

(71) Applicants: Roy LEGUIDLEGUID, Union City, CA (US); Robert Garabedian, Sunnyvale, CA (US); Michael P. WALLACE, Pleasanton, CA (US)

(72) Inventors: Roy LEGUIDLEGUID, Union City, CA (US); Robert Garabedian, Sunnyvale, CA (US); Michael P. WALLACE, Pleasanton, CA (US)

(21) Appl. No.: 14/061,641

(22) Filed: Oct. 23, 2013

Related U.S. Application Data
(60) Provisional application No. 61/717,547, filed on Oct. 23, 2012.
(60) Provisional application No. 61/823,827, filed on May 15, 2013.

(51) Int. Cl. A61B 17/16

(52) U.S. Cl. CPC A61B 17/1671
USPC 606/79

(57) ABSTRACT

Tissue modification devices, methods of using them, and devices, systems and methods of adapting them to form tissue modification devices.

### CROSS REFERENCE TO RELATED APPLICATIONS

This patent application claims priority to provisional patent application No. 61/717,547, titled “SHIELDED SINGLE-WIRE TISSUE MODIFICATION DEVICES,” filed on Oct. 23, 2012; and to provisional application No. 61/823,827, titled “TISSUE MODIFICATION DEVICES,” filed on May 15, 2013. Each of these patent applications is herein incorporated by reference in its entirety.

This patent application may also relate to U.S. patent application Ser. No. 13/757,661, titled “TISSUE MODIFICATION DEVICES AND METHODS,” filed on Feb. 1, 2013, which claims priority to U.S. Provisional Patent Application No. 61/596,045, filed on Feb. 2, 2012, and titled “TISSUE MODIFICATION DEVICES AND METHODS,” each of which is herein incorporated by reference in its entirety.

U.S. patent application Ser. No. 13/757,661 is also a continuation-in-part to U.S. patent application Ser. No. 13/338,103, titled “TISSUE MODIFICATION DEVICES AND METHODS,” filed on Dec. 27, 2011. U.S. patent application Ser. No. 13/338,103 is a continuation-in-part to U.S. patent application Ser. No. 12/773,595, titled “TISSUE MODIFICATION DEVICES AND METHODS,” filed on May 4, 2010, Publication No. US-2010-0274250-A1, which claims priority to U.S. Provisional Application No. 61/175,323, titled “TISSUE MODIFICATION DEVICES,” filed on May 4, 2009; U.S. Provisional Patent Application No. 61/254,638, titled “SPINAL BONE CUTTING DEVICES AND METHODS,” filed on Oct. 23, 2009; and U.S. Provisional Patent Application No. 61/285,188, titled “SPINAL BONE CUTTING DEVICES AND METHODS,” filed on Dec. 10, 2009.

U.S. patent application Ser. No. 12/773,595 is also a continuation-in-part to U.S. patent application Ser. No. 12/496,094, titled “ACCESS AND TISSUE MODIFICATION SYSTEMS AND METHODS,” filed on Jul. 1, 2009, Publication No. US-2010-0004654-A1, now abandoned; which claims the benefit of U.S. Provisional Patent Application No. 61/077,441, titled “INNER SPINOUS DISTRACTION ACCESS AND DECOMPRESSION SYSTEMS,” filed on Jul. 1, 2008.

U.S. patent application Ser. No. 12/773,595 is also a continuation-in-part to PCT Application No. PCT/US09/50492, titled “TISSUE MODIFICATION DEVICES,” filed on Jul. 14, 2009, now Publication No. WO-2010-009093, which claims priority to U.S. Provisional Application No. 61/080,647, titled “TISSUE MODIFICATION DEVICES,” filed Jul. 14, 2008; U.S. Provisional Application No. 61/081,685, titled “TISSUE MODIFICATION DEVICES,” filed Jul. 17, 2008; and U.S. Provisional Patent Application No. 61/163,699, titled “TISSUE MODIFICATION DEVICES,” filed on Mar. 26, 2009.

U.S. patent application Ser. No. 13/338,103 also claims priority to U.S. Provisional Patent Application No. 61/427,432, titled “TISSUE MODIFICATION DEVICES AND METHODS,” filed on Dec. 27, 2010; and U.S. Provisional Patent Application No. 61/472,107, titled “TISSUE MODIFICATION DEVICES,” filed on Apr. 5, 2011.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD

Described herein are systems, devices, and methods of using them, for cutting tissue, and particularly spinal bone and soft tissue in a way that minimizes potential damage to surrounding tissue, including the spinal nerves and vasculature. The methods, devices and systems described herein may be used as part of a spinal surgical procedure involving a complete or partial removal of spinal bone or joint, for example including but not limited to laminectomy, laminotomy, facetectomy, facetolaminotomy, pediculectomy, laminoplasty, corpectomy, spondylectomy, or osteotomy or combinations thereof.

### BACKGROUND

Surgical intervention may require the manipulation of one or more medical devices in difficult to access regions, particularly using minimally invasive or substantially non-invasive techniques. Such interventions may also require manipulations in close proximity to a nerve or nerves, which may risk damage to the nerve tissue or other tissues. For example, medical devices may be used to cut, extract, suture, coagulate, or otherwise manipulate tissue including tissue near or adjacent to neural or vascular tissue. Spinal decompressions are one type of procedure that may be performed to remove tissue that is impinging on a spinal nerve. It would be beneficial to be able to cut or manipulate tissue (and especially bone) in a way that avoids or protects nearby structures such as nerves and blood vessels, while allowing precise removal of bone or portions of bones.

For example, a Transforaminal Lumbar Interbody Fusion (“TLIF”) procedure is a surgical technique to stabilize the spinal vertebra and the disc or shock absorber between the vertebrae. In this (arthrodesis) procedure, lumbar fusion surgery creates a solid structure (bone and/or interbody device) between adjoiningvertebrae, eliminating movement between the bones. The goal of the surgery is to reduce pain and nerve irritation. The procedure typically involves removal of a great deal of spinal bone, e.g., by cutting through the patient's back and removing the facet joints to create an opening into which a spacer or interbody cage can be inserted and filled with bone graft material. Interbody devices such as cages or spacers are often between about 8 mm wide to about 15 mm wide. Pedicle screws and rods or plates may then be used to fix the vertebrae in preparation for a subsequent fusion.

It is common to do a laminectomy as part of the TLIF procedure, in order to provide space for the insertion of the spacer or cage. Other, similar procedures such as Posterior Lumbar Interbody Fusion (PLIF) procedures also involve cutting and removing a region of bone from the spine, such as the removal of a portion of the inferior articulating process (IAP). Removal of these relatively large portions of bone may be difficult, and may require cutting through a substantial amount of otherwise healthy tissue. In addition, the effort of cutting through the bone may damage nearby tissue, including nerve tissue such as nerve roots which are intimately associated with the spine in the dorsal column region being modified. The risks and difficulties of the procedures described above and other such surgical procedure may be exacerbated by the need to make multiple cuts in bone and other tissues, which cuts are typically performed sequentially. In addition, procedures such as these that involve cutting of spinal bone must be performed in difficult to reach regions, and the surgical procedures performed may necessarily need to navigate narrow and tortuous pathways. Thus, it would be of particular interest to provide devices that are relatively low profile, or are adapted for use with existing low-profile surgical devices and systems. It would also be beneficial to provide devices capable of making multiple, simultaneous cuts at different positions in the tissue (e.g., bone).

Described herein are devices, systems and methods that may address many of the problems and identified needs described above.

### SUMMARY OF THE DISCLOSURE

In general, described herein are devices, systems and methods for cutting predetermined regions of tissue, including the spine.

In particular, described herein are wire cutting devices that are configured for easily and reliably cutting bone. In some variations the wire cutting device is optimized for cutting bone using a cable or wire to which a plurality of ferrules have been attached. The ferrules are attached in a configuration that permits optimal control of the device to avoid sticking within the tissue, minimize breakage of the wire, e.g., by fatigue failure, and maximizing efficiency of the cutting. In general, a ferrule may be any structure or element that is placed and attached (e.g., crimped, glued, etc.) onto the wire thereby modifying (e.g., increasing) the wire's diameter over a limited region. A ferrule may be, but is not limited to, a ring, tube, toroid, band, bead, or the like than can be threaded onto a wire. The ferrule it typically rigid, though elastic or elastomeric ferrules may be used in some variations. The ferrule may have a height that is, for example, equal to the radial distance between the inner and outer diameters of the ferrule, or one half the outer diameter from the inner diameter. The ferrule may have one or more edges (e.g., having an anterior annular face and a posterior annular face). In some variations the ferrule is a tubular structure; the tube may have a length that is shorter than the outer diameter.

In general, the devices described herein may be bimanual tissue modification devices having (or usable with) a proximal handle at the proximal end, an elongate shaft connected to the handle, and a cutting region at the distal end. The cutting region may have a single cutting wire (with attached ferrules), or it may have more than one, typically adjacent and/or parallel, wires with ferrules. In some variations the device may include a guidewire coupler at the distal end so that it can couple to a guidewire. For example, a guidewire may be passed through the body in a path that places it adjacent or at least partially around a target tissue (e.g., bone, soft tissue, etc.) to be cut; the distal end of the devices described herein may then be coupled to the guidewire and pulled into position. For example, the proximal end of the guidewire may be coupled to the distal end of the device (via the guidewire coupler), and the device pulled into position by pulling on the proximal end of the guidewire. The end-to-end connection between the guidewire and the distal end of the device may be configured to have sufficient pull strength to support multiple (e.g., tens) of pounds of force so that the guidewire can be used to reciprocate the device at the distal end, while alternately pulling on the proximal handle (or proximal region) of the device.

Some variations of the devices described herein are configured so that they include a backing (backing element, shield, spacer, etc.), which acts as a shield (e.g., protecting non-target tissue from the cutting action of the cutting region of the device), and/or as a spacer (e.g., holding two or more cutting wires spaced apart an appropriate distance until they engage with the tissue to be cut). The backing member/spacer/shield is typically separable from the cutting region of the device and may be configured so that as the cutting member cuts into the tissue, the backing member stays behind (in essentially the path taken by the guidewire) until the cutting is complete. A biasing (e.g., spring) member may be used to allow the relative length of the backing member and bias to increase as the cutting element and the rest of the device cuts into the tissue and is drawn towards the surgeon. Any appropriate bias (which may be referred to as a biasing member, biasing element, spacer bias, backing bias, or shield bias) may be used, and may include a spring, an elastic region, a telescoping region, or the like. In general, the bias may allow extension from a length and be biased to return to that original length, or to an unbiased (shortened) length.

In operation, the use of a biasing member allows the relative length of the path of the backing member to be longer than the length of the path of the cutting member. However, the bias force on the backing may result in a force against the tissue being cut as the cutting element cuts deeper into the tissue (further extending the bias). This may result in “ejecting” the cut tissue from the body at the point at which the tissue is sufficiently cut that it can be removed from the body. This force against the cut tissue may be undesirable. Thus, in some variations, the backing and bias may be configured so that they minimize the biasing force on the backing and therefore the tissue. This may be achieved by configuring the backing and bias so that as the bias extends a certain amount, it is locked and prevent from returning back to the original position. For example, the device may include a plurality of set positions for the extension of the backing (e.g., spacer/shield) as it extends from the cutting region from which it cannot retract back to an initial starting position (or to another set position closer to the starting position). This may be achieved, for example, by one or more tabs or notches on the device, such as on an elongate shaft of the device from which the cutting region extends, that the bias can slide over in a first (e.g., distal) direction, but not backwards over (e.g., proximally). The bias may include a lock such as rigid ring or other structure that slides over the shaft of the device, allowing the bias to slide (with the backing) distally, but only retract back proximally a limited degree. This mechanism may therefore prevent the backing and bias from placing too much force on the tissue during and after cutting.

For example, a tissue modification device may include: a proximal handle; a shaft extending distally from the handle; a bias disposed on the shaft; a cutting wire extending distally from the shaft, the cutting wire comprising a cable and a plurality of ferrules disposed at predetermined locations along the cable; a guidewire coupler at a distal end of the device; and a backing coupled to the bias and extending adjacent to the cutting wire, wherein the backing is configured to separate from the cutting wire when the cutting wire cuts into tissue.

In general, unless the context indicates otherwise, the term “wire” may refer to a single strand wire, a woven, multi-stranded wire, a cable (single or multi-strand), a twisted cable, or the like. For example, a wire may be a cable having a plurality of twisted (single or individual) wires.

Although a ferrule may be connected to a wire in virtually any appropriate manner, in particular, a ferrule may be tubular region that is crimped onto the wire. In particular, a ferrule may be hex crimped to the cable. The ferrule may be circumferentially crimped to the wire. In some variations, the hex crimps are about ⅓ the length of the ferrule. The hex crimp, in which the inner band region of the ferrule is compressed, may cause the outer edges of the ferrule to flair out slightly (“bell outwards”), which may reduce stress concentrations where the crimped ferrule comes into contact with the cable, and may also increase the aggressiveness of the cutting.

As mentioned above, the spacing and/or arrangement of the ferrules on the wire(s) of the devices described herein may be arranged so that they provide advantages. For example, the ferrules may be arranged on one or more (e.g., adjacent) wires so that they provide clear regions at both the distal and proximal (for bimanual devices) regions where there are not any ferrules, allowing the user to relatively easily slide the ferrule-less region of the wire(s) through the tissue before the tissue is contacted by the ferrule region of the cutting member. This proximal and distal region may be referred to as a gap. For example, the cutting wire may include a proximal gap that is free of ferrules and a distal gap that is free of ferrules where the proximal gap and the distal gap are each between about 5 mm to about 25 mm in length. For example, a gap of approximately 13 mm works well.

In addition, the spacing of the ferrules may be configured so that they allow aggressive cutting while being sufficiently easy to pull though the tissue. The inventors have found that for ferrules of size having a wall thickness of between about 0.051 mm and 0.254 mm, spacing the ferrules in a range of between about 0.51 mm and 6.35 mm apart works allows efficient tissue cutting without being too difficult to manipulate. For example, for a wire having an OD of 0.61 mm, and a ferrule of OD 0.914 mm and ID of 0.660 mm (ferrule thickness of approximately 0.127 mm), a ferrule spacing of between about 1 mm to up to about 3 mm (about 0.04″ to about 0.12″), the tactile feel and cutting effectiveness/aggressiveness of the device was good.

In variations including a bias or element, the bias may be positioned at least partially within the handle.

As mentioned above, in some variations, the device including a lock for preventing the bias/element from retracting the backing after it extends beyond a position relative to the rest of the device. For example, the device may include a lock that is slidably disposed with the shaft and attached to a distal end of the bias. The device (e.g., the shaft of the device) may further comprise one or more retention regions configured to engage the lock to inhibit travel of the lock (e.g., a slidable lock) in a proximal direction. In some variations, the distance between the distal end of the shaft and the distal end of the bias is at least about 60 mm.

As mentioned above in some variation, the devices may include two (or more) adjacent and/or parallel cutting wires. A second wire may also include ferrules, which may (but do not have to) be arranged identically to those on a first wire. For example, the device may include a second cutting wire extending distally from the shaft adjacent to the cutting wire. The first and second wires may be different regions of the same wire, which may wrap around one or both ends of the device (e.g., the distal end) or they may be separate wires.

In some variations, the backing may be configured as a spacer that releasably holds the cutting wires, e.g., a first cutting wire and a second cutting wire, a distance from one another. The backing may be configured as a shield.

For example, described herein are elongate, bimanually controlled tissue modification devices for cutting tissue in a patient, the device comprising: a pair of flexible, elongate cutting members extending along an elongate length of the device; a spacer, wherein the spacer is sized and configured to operate in one of two modes: a first mode, wherein the spacer is coupled to the cutting members such that it holds a portion of each of the two cutting members a distance from one another, and a second mode, wherein at least a portion of the spacer is moved away from a cutting member to allow the cutting members to cut further into tissue; and a bias connected to a proximal end of the spacer and a proximal region of the device, wherein the bias is configured to extend the proximal end of the spacer distally as the spacer is moved away from the cutting member.

As mentioned, the device may further comprise a lock configured to prevent the bias from retracting the proximal end of the spacer proximally once the bias is extended distally past a predetermined region on the device. In some variations, the device further includes an at least one lock configured to prevent the bias from retracting the proximal end of the spacer proximally once the bias is extended distally past a plurality of predetermined regions on the device. The lock comprises a ring coupled to the bias and configured to engage one or more tabs on a proximal shaft of the device.

Also described are tissue modification devices that may include: a proximal handle; a shaft extending distally from the handle; a pair of cutting wire extending distally and adjacent to each other from the shaft, at least one of the cutting wires comprising a cable and a plurality of ferrules disposed at predetermined locations along the cable; a guidewire coupler at a distal end of the device; and a spacer configured to hold the cutting wires a distance from one another and release the cutting wires as the cutting wires cut tissue.

Also described herein are apparatuses (e.g., devices and systems) and methods of using them for cutting predetermined regions of tissue, such as a spine.

For example, described herein are single-wire tissue modification devices configured to cut target tissue including bone and ligament. These single-wire tissue modification devices may be joined (e.g., using an adapter) to form a tissue modification device having two or more cutting wires. Any of the wire cutting devices described herein may be connected together in parallel.

Described herein are single-wire tissue modification devices including: an elongate length of cutting wire extending proximally to distally; a guidewire coupler coupled to the distal end of the cutting wire; a proximal handle coupled to the cutting wire; and a shield (e.g., backing) at least partially surrounding the elongate length of the cutting wire, wherein the cutting wire is configured to be exposed from the shield during use.

The device may also include a slit extending along a length of the shield proximally to distally, wherein the cutting wire is configured to pass out of the shield though the slit during use. In some variations, the shield comprises a removable material (e.g., paraffin such as bone wax, etc.).

Any of these devices may also include a tensioning element coupled to an end of the shield and configured to extend the length of the shield relative to the cutting wire during use.

The shield may include one or more cut-out regions, such as a plurality of cut-out regions, extending along the length of the shield on an upper surface of the shield. These cut-out regions may enhance flexibility and may help orient the device. Further, a slit and/or other cut-out regions may expose at least a portion of the cutting wire, and allow the cutting wire to cut into the tissue (e.g., bone), displacing the shield away from the cutting wire as the cutting wire enters the tissue. Thus an opening (e.g., slit, cut-out region) in the shield may allow the cutting wire to exit the shield so that it is left behind as the cutting wire is cut laterally through the tissue.

In some variations, the shield may be configured as a sheath. The sheath may be coaxial with the cutting wire, and removed from the cutting wire (or applied to the cutting wire), once the cutting wire is passed through the tissue (e.g., the foramen) and positioned to cut.

A tensioning element may be a spring coupled to the proximal end region of the shield, or other biasing element. In some variations, the distal end region of the shield is coupled to the distal end region of the cutting wire. In some variations, the spring could is positioned at the distal end, and the proximal end may be fixed.

Also described herein are single-wire tissue modification devices comprising: an elongate length of cutting wire extending proximally to distally; a guidewire coupler coupled to the distal end of the cutting wire; a shield at least partially surrounding the elongate length of the cutting wire; an exit region extending along a length of the shield, wherein the cutting wire is configured to pass out of the shield though the exit during use; and a tensioning element coupled to an end of the shield and configured to extend the length of the shield relative to the cutting wire during use.

The exit region may comprises a slit, or a pre-formed (e.g., perforated) region. The device may further include a plurality of cut-out regions extending along the length of the shield on an upper surface of the shield.

The tensioning element may include a spring coupled to the proximal end region of the shield.

In some variations, the distal end region of the shield is coupled to the distal end region of the cutting wire.

Any of the single-wire tissue modification devices described herein may be used with an adapter device for forming a tissue modification device comprising a pair of parallel cutting wires. In general, an adapter may include a spacer comprising a pair of adjacent hitch regions at a proximal end region of the spacer, each configured to mate with a guidewire coupler at the distal end of a single-wire tissue modification device, wherein the hitch regions are separated by a predetermined amount; and a guidewire coupler at a distal end of the spacer, the guidewire coupler configured to couple with the proximal end of a guidewire to pull the coupler into position through the tissue.

In some variations, the adapter further comprises a pair of stimulation electrodes on an upper surface of the spacer, the electrodes adapted to provide bipolar stimulation to detect a nerve or nerves near the upper surface. The adapter may also include a second pair of stimulation electrodes on a lower surface of the spacer, the electrodes adapted to provide bipolar stimulation to detect a nerve or nerve near the lower surface.

Methods of cutting tissue using any of the devices described herein are also included. For example, described herein are methods of cutting a target tissue comprising: positioning a guidewire in a curved path immediately adjacent to a target tissue; coupling the distal end of a single-wire tissue modification device to the proximal end of the guidewire; pulling the guidewire distally to position the single-wire tissue modification device adjacent to the target tissue; and reciprocating the single-wire tissue modification device by alternately pulling the proximal and distal ends of the single-wire tissue modification device so that a cutting wire of the single-wire tissue modification device exits a shield of the single-wire tissue modification device and cuts into the target tissue, leaving the shield outside of the cut region.

The method may also include applying tension to the shield to drive it against the cut region of the target tissue. In some variations, the method includes confirming that a nerve is not present between the curved path and the target tissue prior to reciprocating the single-wire tissue modification device. Finally, the method may include removing the single-wire tissue modification device after cutting the target tissue by cutting the shield and cutting wire.

### BRIEF DESCRIPTION OF THE DRAWINGS

FIGS. 1A-1D illustrate elements that may be used for performing the methods described herein; some or all of these elements (the guidewire of FIG. 1A, neural localization device of FIG. 1C, flexible wire saw of FIG. 1B, and parallel wire saw of FIG. 1D) may form a system for cutting tissue.

FIGS. 2 and 3A-3D show various examples of a cutting wire.

FIGS. 3E1, 3F1 and 3G1 shows examples of ferrules that may be used to form a cutting wire; FIGS. 3E2, 3F2 and 3G2 illustrate cutting wires using each of these, respective, ferrules.

FIGS. 3H1-3H3 illustrate another example of a ferrule that can be used in a cutting wire.

FIG. 3H4 shows one variation of a device having two adjacent cutting wires with ferrules similar to those shown in FIGS. 3H1-3H3; the cutting wires are held a predetermined distance apart at both proximal and distal ends of the tissue modification (cutting) region.

FIG. 3H5 shows another variations of an elongate, bimanually controlled tissue modification device for cutting tissue in a patient in which the cutting wires include gap regions at the proximal and distal regions of the tissue modification (cutting) region.

FIG. 4 illustrates a tissue modification device having a cutting wire.

FIG. 5 illustrates a method for cutting a lateral portion of a facet.

FIG. 6A illustrates a variation of a method for cutting the lateral portion of a facet.

FIG. 6B illustrates one step of a method for cutting a facet.

FIG. 7A shows a side view of one variation of a single wire shielded cutting wire; FIG. 7B shows a side view of one variations of a single wire shielded cutting wire.

FIG. 7C shows a proximal portion of one variation of an elongate, bimanually controlled tissue modification device for cutting tissue in a patient, including a handle into which the bias (shown in this example as a spring) is housed at least partially within the handle, which is shown in cross-section.

FIG. 7D shows a mid-proximal region of an elongate member of an elongate, bimanually controlled tissue modification device for cutting tissue in a patient; the elongate member shows two locking region (tabs) that may engage with a lock (ring) connected to the bias to prevent the bias from retracting the backing (not shown) to the initial proximal position of the bias.

FIGS. 7E and 7F illustrate the mid-proximal region of an elongate, bimanually controlled tissue modification device for cutting tissue in a patient; the device include a bias (spacer bias) that is in an initial, proximal position, holding the spacer/shield (e.g., backing) so that it is adjacent to the two adjacent cutting wires.

FIGS. 7G and 7H illustrate the same device shown in FIGS. 7E and 7F in which the cutting wires have been moved away from the spacer while the spacer is allowed to remain behind after disengaging from the cutting wires, as would occur when the cutting wires cut into tissue. The bias is extended distally and a lock (in this example a sliding ring at the distal end of the bias) has engaged a retention region (e.g., tab) on the rigid elongate member between the handle and the cutting region.

FIGS. 7I and 7J illustrate the device of FIGS. 7E-7F and 7G-7H in which the cutting wires have been moved even further away from the spacer, e.g., into the tissue, and the bias is extend even further distally and engaged at a more distal retention region.

FIG. 8A shows a section though one variation of a shielded region of a shielded cutting wire. FIG. 8B shows a portion of a shielded cutting wire. FIG. 8C shows an alternate version of a portion of a shielded cutting wire.

FIG. 9A shows a middle region of one variation of a shielded cutting wire; FIG. 9B shows a distal end region of the shielded cutting wire; FIG. 9C shows an enlarger region of the distal end of the shielded cutting wire, including a guidewire coupler.

FIGS. 10A and 10B show the proximal region of one variation of a shielded cutting wire having a bias connected to the shield. FIG. 10C shows an enlarged view of the proximal region, including the bias.

FIGS. 11A-11D show variations of shield elements coupled to cutting wires.

FIGS. 12A-12E illustrates the operation of different variations of shielded cutting wires cutting bone.

FIGS. 13A and 13B show another variation of a shielded cutting wire, including a wearable material forming the shield, in side and cross-sectional views.

FIGS. 13C-13E show another variation of a shielded single-wire tissue modification device.

FIG. 14A shows one variation of coupler for forming a two-wire tissue modification device by coupling to two individual cutting wires. FIG. 14B shows a side perspective view of a coupler as shown in FIG. 14A. FIG. 14C shows an example of a handle coupler that may be used with this system.

FIG. 15 shows another variation of a coupler for forming a two-wire tissue modification device including a nerve stimulation region for checking proximity to a nerve.

FIGS. 16A-16C illustrate one variation of a tissue modification device configured as a facetectomy device. FIG. 16A illustrates the entire device from the proximal end to the distal end. FIG. 16B shows an enlarged view of region B of FIG. 16A, showing the flexible distal end region including a cutting region (region C) and non-cutting regions. FIG. 16C is an enlarged view of cutting region (region C) showing spacers separating cutting rungs from which box-like cutting teeth that taper at their base project.

FIGS. 17A, 17B and 17C show enlarged views of the cutting region of the device of FIG. 16A, showing the cutting rungs having cutting teeth with tapered bases separated by spacers; the cutting teeth are arranged on the cutting rungs so that a gap region is present in an alternating locations at adjacent cutting rungs. FIG. 17A is a top perspective view. FIG. 17B show a back view of the cutting region, including a smooth surface composed of the smooth surface of the back surface of the cutting rungs and the intermediate ferrules. FIG. 17C is a front perspective view.

FIG. 18A shows a front view of the distal end of the device of FIG. 16A (including the end of the device from the cutting region distally). The cutting teeth have a tapered base that is narrower than the top region opposite the base, so that the teeth extend beyond the edge of the base, as shown.

FIG. 18B shows a front view of one variation of a cutting rung of the device of FIG. 16A.

FIG. 18C illustrates a side view of the same rung of FIG. 18B.

FIG. 18D shows a front perspective view of the rung of FIG. 18B.

### DETAILED DESCRIPTION

Described herein are devices, systems and methods for cutting spinal tissue such as bone and/or soft tissue, and particularly spinal bone in the dorsal column using a flexible cutting element that may be passed around the bone.

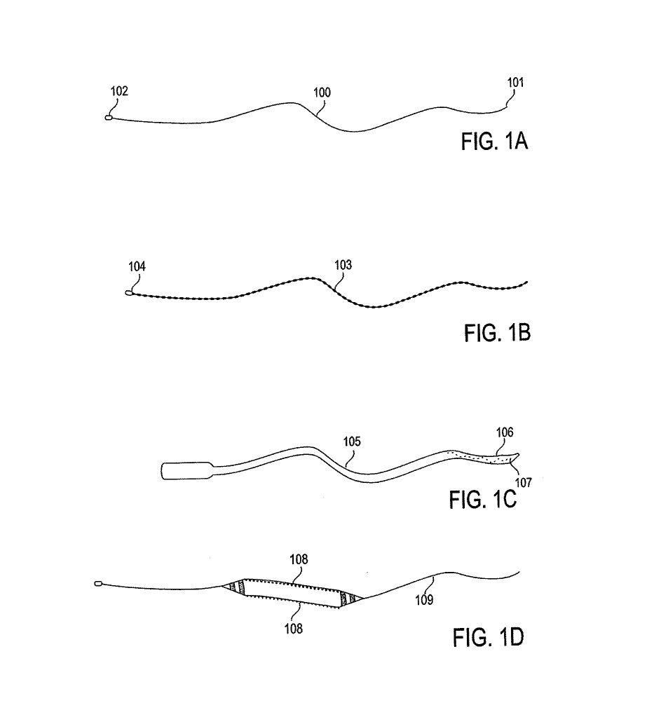

The methods, devices and systems described herein may be used as part of a spinal surgical procedure involving a complete or partial removal of spinal bone or joint, such as a laminectomy, laminotomy, fascetectomy, pediculectomy, etc. FIGS. 1A-1D illustrate different elements that may be used as part of a system for cutting bone as described. FIG. 1A shows a guidewire 100 that is adapted to couple to the distal end of another device so that the device may be pulled into position using the guidewire. The distal end 101 of the guidewire may be sharp, and the proximal end may include a coupling joint 102 (e.g., a ball or other enlarged region that can be gripped by a coupling member). FIG. 1B shows one variation of an elongate cutting member, such as a cutting wire 103 having an abrasive surface for cutting tissue, such as soft tissue (e.g. ligament) and/or bone. Saw wires (e.g., traditional Gigli saws) may be adapted for use with a guidewire as described herein. For example, the distal end of the saw may include a guidewire coupler 104. In some variations the wire is thin or flattened. Any appropriate saw or cutting element may be used, including those that cut by reciprocation or by application of energy.

FIG. 1C illustrates one variation of a flexible ribbon-shaped neural localization device 105. The distal end 106 of the device is substantially flat (not apparent from the figure) and has flat sides with one or more electrodes 107 along the surface to stimulate a nerve or neural tissue, if nearby.

FIG. 1D illustrates another variation of a bone saw device having two parallel cutting wires 108 that are separated by a predetermined distance (e.g., 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, between about 4 mm and about 20 mm, between about 4 mm and about 18 mm, etc.). In some variations, as described herein, the two parallel cutting wires may be any of the single-wire shielded cutters described herein. In some variations, these single wire cutters are coupled to an adapter at one or both ends that holds them separate from each other.

The distal end of the device may be configured for coupling to a guidewire 100. Alternatively, the device may have an integral flexible guide region 109 at the distal end of the device so that the device does not need any additional guidewire/coupler. For example, flexible guide region 109 is shown having a curved shape to demonstrate that at least a portion of flexible guide region 109 may be flexible. The distal portion is preferably flexible in at least one direction, such that it may wrap around a target tissue, while having sufficient column strength such that the distal end may penetrate tissue without buckling. In some examples, the distal end may have a sharp distal tip configured to penetrate and/or pierce tissue. In various examples, flexible guide region 109 may have one or more of a round, ovoid, ellipsoid, flat, cambered flat, rectangular, square, triangular, symmetric or asymmetric cross-sectional shape. Distal flexible guide region 109 may be tapered, to facilitate its passage into or through narrow spaces as well as through small incisions on a patient's skin. Distal flexible guide region 109 may be long enough to extend through a first incision on a patient, between target and non-target tissue, and out a second incision on a patient. In some examples, the distal end may have a length greater than or equal to 3 inches (e.g., 76.2 mm) such that it may extend from around the proximal end of the stimulation region to outside the patient where it may be grasped by a user and/or a distal handle. In some alternative examples, the distal end may have a length greater than or equal to 10 inches (e.g., 254 mm) while in some other alternative examples, the distal end may have a length greater than or equal to 16 inches (e.g., 406.4 mm). Alternatively, distal flexible guide region 109 may be long enough to extend through a first incision, between the target and non-target tissue, and to an anchoring location within the patient.

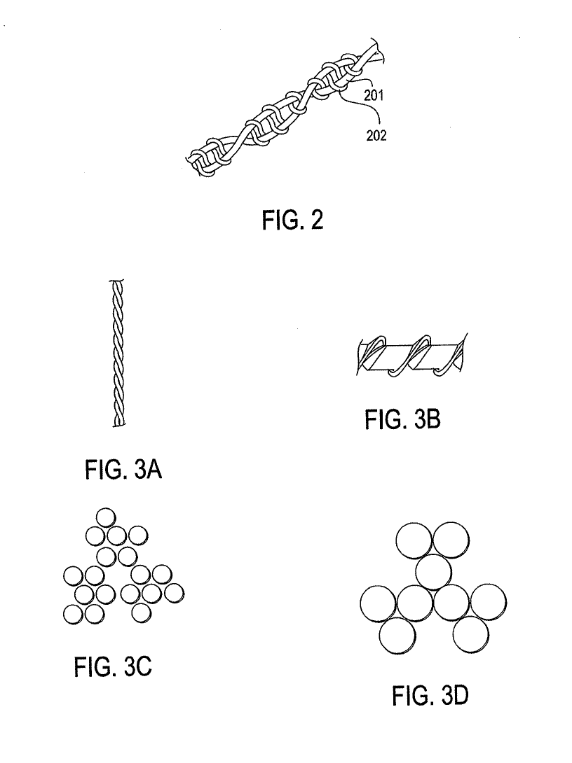

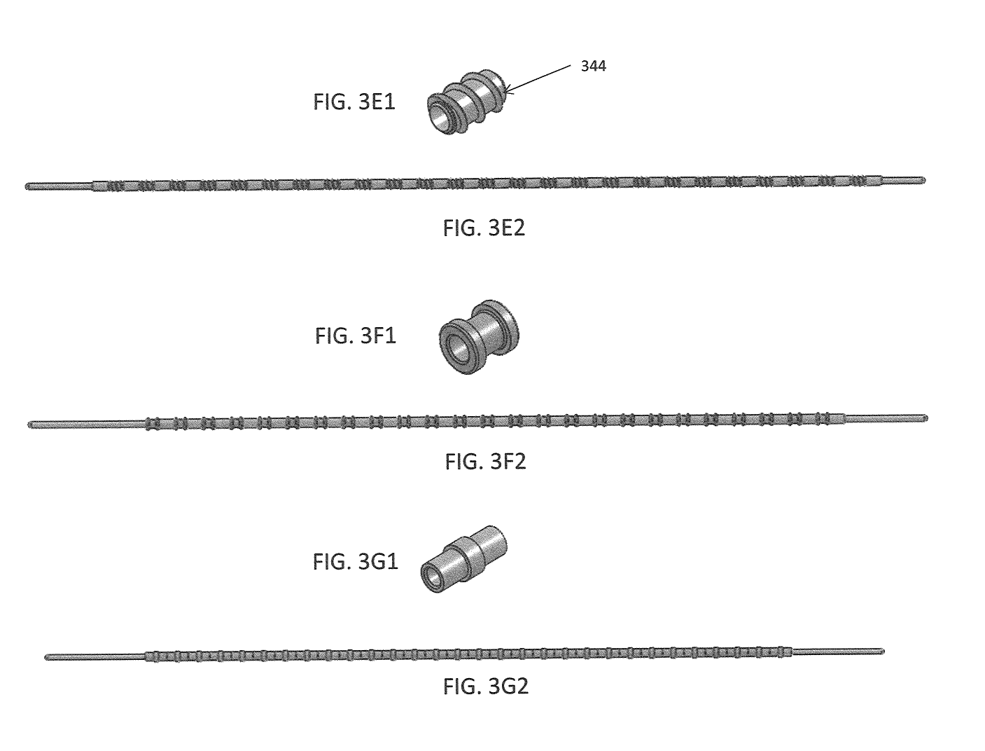

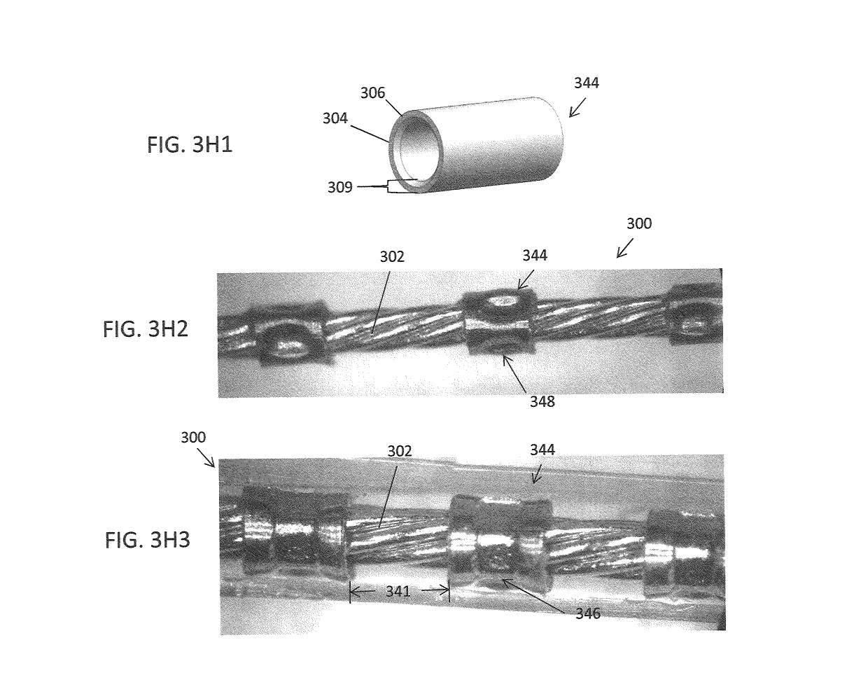

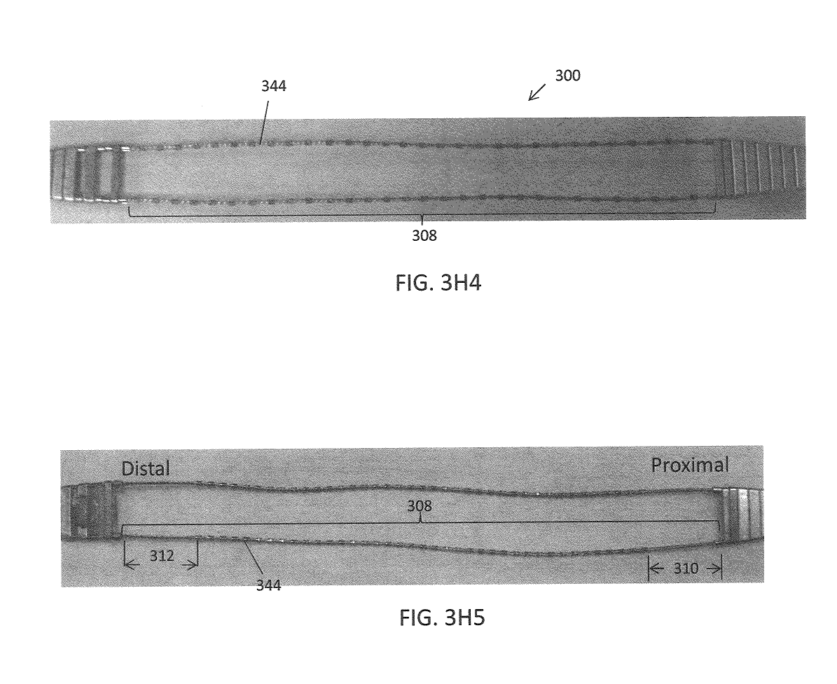

The cutting wires 108 as described herein may be one of several variations of cutting wires, including the shielded cutting wires described herein. In some examples, the cutting wires may have an outer diameter that ranges from 5 to 50 thousandths of an inch, for example (e.g., 0.127 mm to 1.27 mm). A single wire saw may include a plurality of wires wrapped around each other at differing pitches. As shown in FIG. 2, wire 202 having a first diameter, may be wrapped around wire 201 having a second diameter. As shown, the first diameter may be less than the second diameter. FIG. 2 illustrates one example of a Gigli cutting wire. Alternatively, as shown in FIGS. 3A-3D, the cutting wire of the tissue modification device can be one of several alternative examples. For example, as shown in FIG. 3A, the cutting wire may be a second example of a conventional Gigli wire. A Gigli wire is typically made of a first wire having a first diameter wrapped around a second wire having a second diameter. Typically, the second diameter is larger than the first diameter, but alternatively, they may have substantially the same diameter, as shown in FIG. 3A. In some examples, a first set of first and second wires may be wrapped around a second set of first and second wires. Alternatively, as shown in FIG. 3B, the cutting wire may include a single wire that is machined or modified to include a helical or spiral cutting edge along the length of the wire. This wire may be created by cutting or forming a spiral or helical groove along the length of the wire. As shown in FIGS. 3C and 3D, the cutting wires may be formed by winding bunches of wires. For example, as shown in cross section in FIG. 3D, the cutting wire may comprise a 3 by 3 construction. In this example, three wires are first wrapped around one another to create a first bunch of wires. Then three sets of those three wire bunches are subsequently wrapped around each other. As shown in cross section in FIG. 3C, rather than initially wrapping three wires together, 6 wires may be wrapped together to create a bunch of wires and then three sets of the 6 wire bunches may be wrapped together to form a 3 by 6 configuration. Each 6 wire set may be formed as a 7 wire set might be formed, but leaving one wire position empty. In some variations, the cutting wire comprises individual blades that are threaded over a cable. The cable construction may allow for greater fatigue resistance over a single wire or twisted wire construction. For example, FIG. 3E1 shows one variation of a blade (ferrule 344) than can be threaded over a cable as illustrated in FIG. 3E2; multiple ferrules/blades are threaded, so that the cutting wire formed remains flexible. Other cutting ferrules such as those shown in FIGS. 3F1 and 3G1 may also be used, as illustrated in FIGS. 3F2 and 3G2.

FIGS. 3H1-3H5 illustrate another example of a cutting wire 300 having a plurality of ferrules 344 that have been threaded onto a twisted wire cable 302, so that they may be affixed onto the wire. As illustrated in FIG. 3H1, the ferrule 344 can be a cylindrical structure with cutting edges 304 and surfaces 306 at both its proximal and distal ends. Any of the ferrules and cables disclosed herein can be interchanged with the ferrule and cable specifically described herein, including those shown in FIGS. 3H1-3H5. The angle and length of the cutting edges 304 and surfaces 306 and the overall sizes of the ferrule 344 and cable 302 can be varied to provide a balance between cutting efficiency and resistance to cutting and durability. The cutting wires 300 described herein can be used in either a single wire cutting device or a cutting device with two or more cutting wires.

In some examples, the cable 302 can have an outer diameter (OD) of approximately 0.610 mm, or between about 0.254 mm to about 1 mm in 0.025 mm increments. In some examples, the cable 302 can have an OD that is less than or equal to approximately 0.61 mm, or less than or equal to approximately 0.254 mm to about 1 mm in 0.025 mm increments. In some examples, the ferrule 344 can have an OD of approximately 0.914 mm, or between about 0.51 mm to about 1.5 mm in 0.025 mm increments. In some examples, the ferrule 344 OD is about 0.305 mm greater than the cable OD, or between about 0.127 mm to about 0.635 mm in 0.025 mm increments greater. In some examples, the ferrule 344 inner diameter (ID) can be slightly greater than the cable OD. For example, the ferrule 344 ID can be about 0.051 greater than the cable OD, or between about 0.025 mm to about 0.076 mm greater. In some examples, the ferrule 344 ID can be about 0.66 mm. In some examples, the wall thickness (T) 309 of the ferrule 344 can be about 0.127 mm, or between about 0.051 mm to about 0.25 mm, in 0.25 mm increments. A thinner ferrule wall may have a greater tendency to crack when crimped to the cable 302, while a thicker wall, which increases the ferrule OD to cable OD difference, may increase the tactile feel and aggressiveness of the cutting, which may also increase the resistance to cutting. In general, as the cable 302 and ferrule 344 diameter increases, the durability and fatigue resistance under normal loads all may increase as well. However, in general, the cutting effectiveness or efficiency may decrease as the diameter of the cable and ferrule increase.

The ferrule 344 can be fastened to the cable 302 at predetermined locations and along the cable 302 by any appropriate method, including by crimping the ferrule 344 to the cable 302. In some examples, the ferrule 344 may be hex crimped 346 or indent crimped 348 onto the cable 302. In some examples, the hex crimp 346 may be about ⅓ the width of the ferrule 344, which may cause the outer edges of the ferrule 344 to bell outwards thereby increasing the effectiveness and aggressiveness of the cutting while also reducing the stress concentrations where the crimped ferrule comes into contact with the cable. A hex crimp may be used to ensure even pressure is applied to the cable, which may maximize the holding strength to prevent migration while under use. Other techniques of fastening the ferrule 344 to the cable 302 can be employed, such as welding or brazing the ferrule 344 to the cable 302. In comparison to a spiral cut wire, the cable 302 and ferrule 344 based cutting wire 300 may provide improved kink resistance, cutting efficiency, and durability. The spacing 341 between the ferrules 344 may be adjusted to vary the tactile feel and cutting effectiveness and aggressiveness of the cutting wire 300. In some examples, the spacing between the ferrules 344 can be between about 0.50 mm to 6.35 mm in approximately 0.13 mm increments. In some examples, the spacing 344 can be about 1.02 mm to provide a good balance of tactile feel and cutting effectiveness.

In some examples, as illustrated in FIG. 3H4, the ferrules 344 can run the full length of the blade section 308 of the cutting wire 300. In other examples, as illustrated in FIG. 3H5, the blade section 308 has a proximal gap 310 and a distal gap 312 that is a cable portion free of ferrules 344. The proximal gap 310 and the distal gap 312 may be between about 5 mm to about 25 mm in 1 mm increments. In some examples, the gaps 310, 312 can be about 13 mm. The gaps 310, 312 reduce catching during the transition between back and forth strokes by reducing the resistance of the cable at the gaps and allowing the cable to achieve an adequate velocity before the ferrules reengage the bone.

As shown in FIG. 4, a tissue modification device 400 includes a tissue modification portion 401, a distal end 402, and a proximal end 403. The tissue modification portion may include a wire saw configured to cut through bone and/or soft tissue, including the shielded wire saws described below. The distal end 402 may include a guidewire coupler. The proximal end 403 may be more rigid than the flexible tissue modification region 401. The proximal end may be coupled to a proximal handle used to grasp, position, and/or reciprocate the tissue modification device.

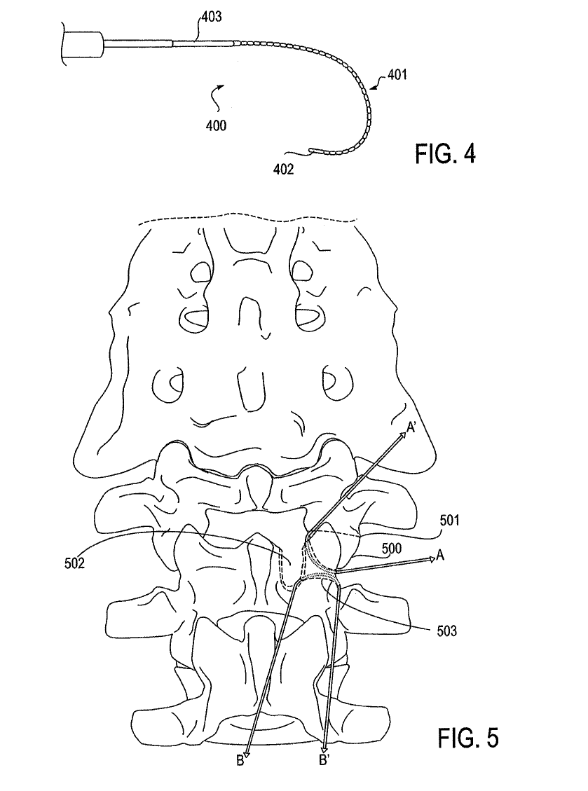

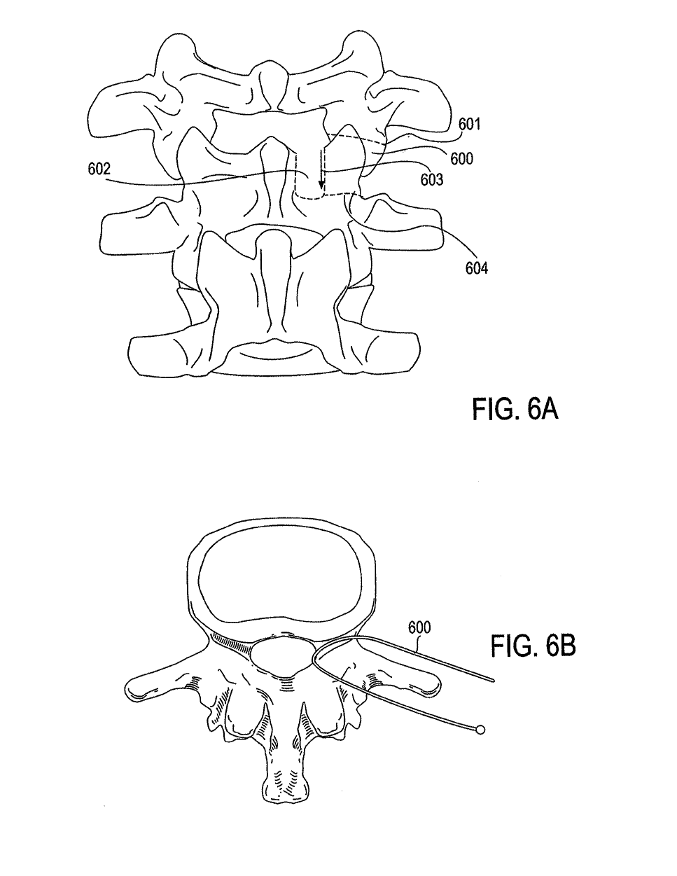

In operation, the devices described above may be used as part of a system for cutting bone, as illustrated in FIGS. 5-6B. In this example, the facet is cut laterally, which may be used as part of a TLIF procedure, for example. FIG. 5 shows an overview of this method. In this example, two cuts through the bone are made sequentially. To cut the facet joint as desired for this procedure, a guidewire is first positioned partially around/against a superior articular process (SAP) 500 that will be cut. The guidewire may be positioned using or more probes or cannula so that the guidewire enters laterally from a first position (A) passes through the foramen and around the SAP 500, and leaves the spine (and exits the patient) at a second position (A′). Once the guidewire is positioned, it may be used to pull in a neural localization device to confirm that a nerve (spinal nerve or nerve root) is not between the bone to be cut and the pathway of the guidewire. After confirmation that the nerve is not between the bone and the pathway, a cutting element (e.g., wire saw IB) may be pulled into position so that both the distal and proximal ends of the wire saw exit the patient and may be pulled upward (in the posterior direction) and reciprocated laterally to cut the SAP as indicated by the dashed line 501 in FIG. 5.

Once the SAP has been cut by the saw, the saw may then be positioned for the second cut, through the lamina. Optionally, before this cut is made, and before the wire saw is moved to this location, the lamina may be prepared by notching or biting away portion of the lamina 502 with a Rongeur or other device (e.g. forming a laminotomy). For example, in FIG. 5, the dotted region shows an area 502 that may be bitten away to form a partial window into the lamina. Once that is done, the cutting element (wire saw) is moved in the cephalid direction (down in the orientation of FIG. 5), so that the ends of the wire extend in the cephalid direction (and across the central spinal axis). Thus, one end of the saw B is present in the notch or window formed in the lamina, and the other end B′ moves along the foramen. If desired, the neural localization device may again be used to confirm that a nerve will not be cut from this position, e.g., by retracting the guidewire proximally, removing the cutting element and attaching the neural localization device. Finally, the cutting element may be reciprocated to cut through the pars region of the lamina along dotted line 503. Thereafter the cut away portion of the bone may be removed, and other procedures performed as desired.

In the examples including two parallel wires, the facet or target tissue suture may be cut in a single step. For example, to perform a Facetectomy, the device may be deployed just cephalad of the caudal pedicle. The parallel wires may be held at the desired width, or the wires may be expanded to the desired width. The desired width may range from 6 mm-15 mm, depending on the interbody device to be inserted between the vertebras, for example. In some variations, the cephalad wire may be expanded to the desired width. The device may then be reciprocated across the tissue to cut through and remove at least a portion of the width of the facet joint. The device may be reciprocated by alternatively pulling a proximal end of the device (e.g. proximal handle) and pulling a distal end of the device (e.g. a distal handle and/or or guidewire). While one end is pulled, the other end may also be pulled to maintain tension across the device.

FIGS. 6A and 6B illustrate alternative views of the steps described above. For example, FIG. 6A shows the steps of cutting through the SAP 600 (dotted line 601), biting away/forming a window 602 in the lamina, moving the cutting element in the caudal direction (arrow 603) and cutting the pars (dotted line 604). FIG. 6B shows a cross-sectional view through the spine indicating the cutting of the SAP. As shown, the cutting wire 600 is positioned through the foramen and around the SAP (not shown). The wire will be reciprocated and pulled in the direction of the arrows to cut through the SAP.

As mentioned above, in any of the facet joint procedures described herein, all or a portion of the facet (e.g., the superior and/or inferior spinous processes) may be cut. For example, a procedure for fusing or preparing a facet joint may include a facetectomy, particularly for TLIF (Transforaminal Lumbar Interbody Fusion) procedures. The procedure may include a facet joint treatment device that is configured to saw through bone. For example, the device may include one or more cable-type saws including a distal end that is configured to couple to the pull wire as described above. As mentioned, a probe or probes may be used to place the pull wire under the facet joint. A facet joint modifying device may then be pulled in under bimanual control. Pulling the facet joint modifying device dorsally (e.g., by distal/proximal reciprocation) would result in the removal of the entire facet joint. This method may be faster than current methods which involve slow biting with Rongeur-type devices.

### Shielded Single-Wire Tissue Modification Devices

A shielded single-wire tissue modification device may include a tissue cutting wire extending from a proximal handle to a distal guidewire coupler, and a tissue-protecting shield or sheath may cover at least a portion of the length of the tissue cutting wire. The shield may include a longitudinal opening, slit, perforation, or the like, from which the tissue cutting wire may extend as it cuts into the tissue (e.g., bone), leaving the shield behind. The longitudinal slit or opening may be configured so that it along the length of the device for the majority of the length of the tissue cutting wire, or for a portion of the length of the tissue cutting wire. Alternatively, in some variations described in greater detail below, the shield is a coating or layer that is worn away to expose the cutting wire on one or more sides or regions of the wire.

A shielded single-wire tissue modification device is typically configured to be operated by reciprocating and pulling the proximal and distal ends of the device so that the cutting wire of the device cuts through the tissue. The shield is separated from the cutting region as it is pulled through the tissue. In some variations, the shield is left behind (typically out of the region being cut); in other variations the shield is worn away from the cutting wire, exposing the cutting surface.

In general, the shield may completely or partially cover the cutting wire when the device is in the resting state (e.g., not under tension). In some variations, the shield is configured to include cut-out regions along at least one side (e.g., the “upper side” or same side on which the slit/opening/perforation is located). Cut-out regions may enhance flexibility of the device. In some variations the cut-out regions form windows along the length of the upper surface of the device.

In use, the cutting wire may separate from the shield during operation, and the shield may be left outside of the cut region of the tissue as the cutting wire passes through the tissue being cut. For example, the distance between the handle and the guidewire connector traveled by the shield may be longer than the distance between the handle and the guidewire connector traveled by the cutting wire. However, it would be beneficial to keep both the cutting wire and the shield in tension. Keeping the cutting wire in tension may allow it to efficiently cut through the tissue (e.g., bone, ligament, etc.). Keeping the shield in tension may prevent the shield from pushing on the neural or other adjacent tissues opposite the cutting region.

Thus, the shielded single-wire tissue modification devices (“single wire cutting devices”) described herein may be configured to maintain tension on both the cutting wire and the shield even as the cutting wire separates from the shield. Put slightly differently, the single-wire devices described herein may maintain tension on both the cutting wire and the shield even as the distance along the paths between the handle and the distal end of the device (e.g., the guidewire coupler) traversed by the cutting element and the shield differ. For example, in some variations the shield may be elastomeric and/or may include a biasing region (e.g., a spring at the proximal or distal region), and/or the shield may be configured to slide relative to a proximal or distal end region. Examples of these variations are described in greater detail below.

Singe-Wire Tissue Modification Devices with Tensioning Element

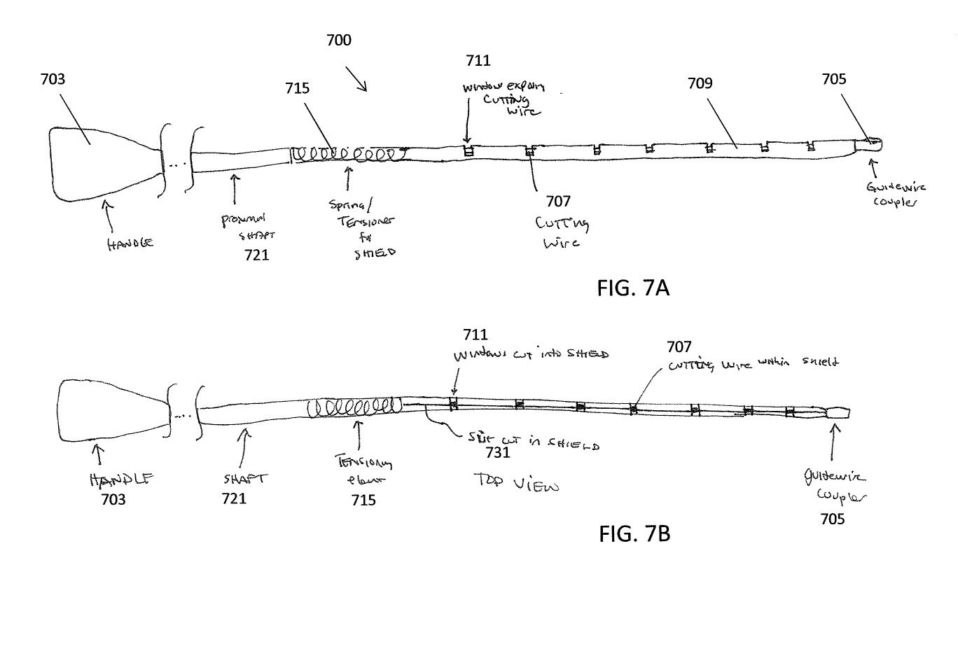

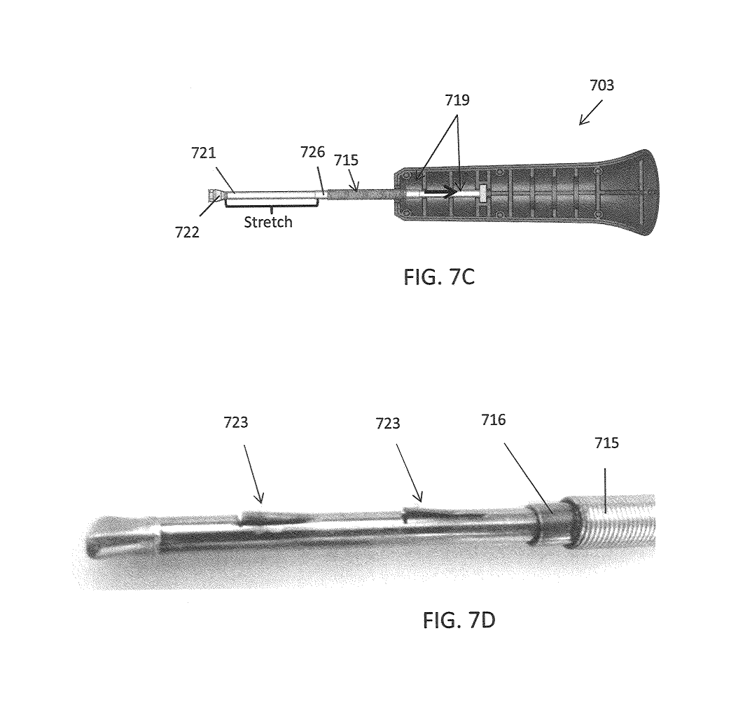

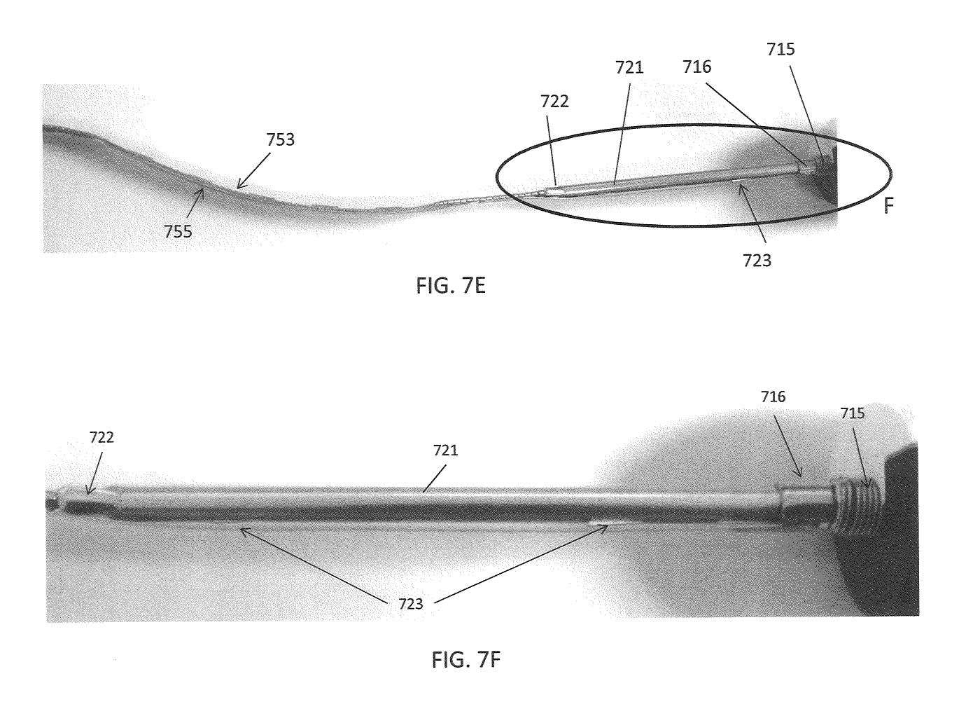

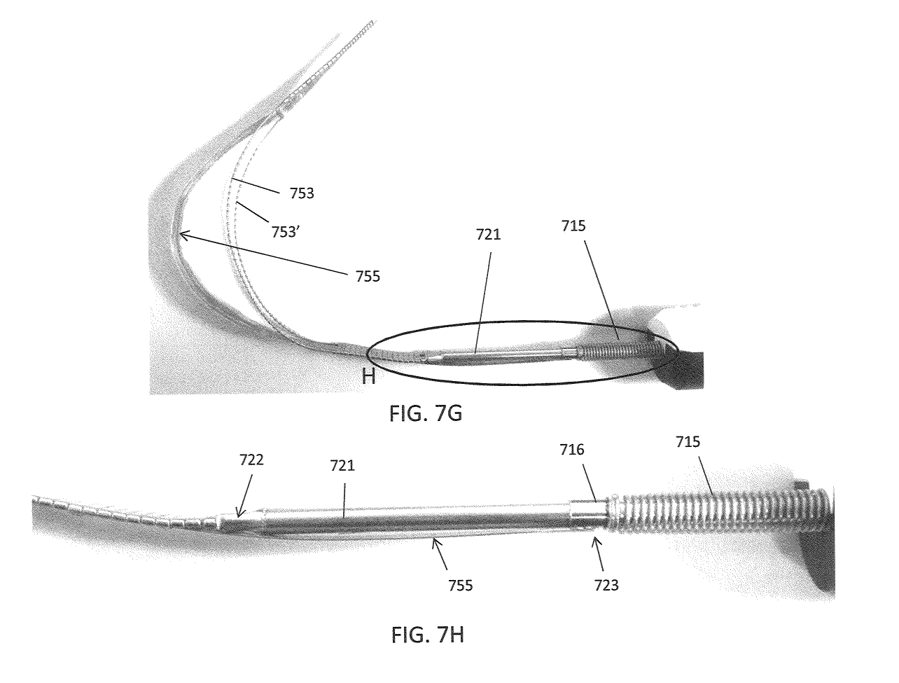

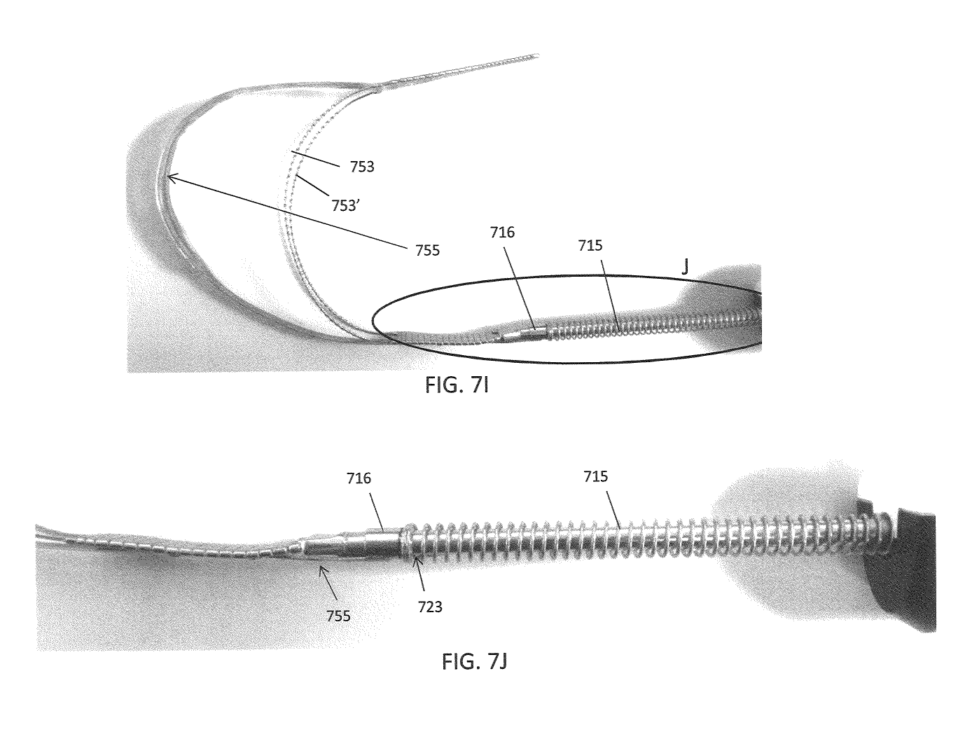

FIGS. 7A and 7B illustrate one variation of a shielded single-wire tissue modification device 700 including a proximal handle 703, a distal guidewire coupler 705, a cutting wire extending along the length between the proximal handle and the distal guidewire coupler (where the cutting wire may be any variation of a cutting wire as described above), and a shield 709 at least partially covering the cutting wire. As mentioned above, the figures are not shown to scale. For example, in FIGS. 7A and 7B, the slit section may be smaller than shown, and the cut-out region may be larger than shown.

The shield is generally configured to flex and bend as the cutting wire is bent. For example, the shield may be flexible material, and/or may include regions allowing it to more readily bend or flex. For example, the shield may be configured to include cut-out regions 711 or hinged regions. In some variations the material properties are sufficient to allow the shield to bend or flex in at least one plane. The shield may completely surround the cutting wire, or it may surround it on at least one side (the shielded side). In general, the shielded single-wire tissue modification device may include an orientation having an upper (cutting direction) region opposite to a lower (shielded direction) region.

At least the portion of the shield facing the lower direction may be configured to be atraumatic for the tissue, and may be smooth. The tissue-facing surface of the shield may also be configured to slide or move over the tissue without substantially damaging it. For example, the surface may include a lubricant. The shield may be made of a polymeric material (e.g., PEEK, etc.) which may include an additional surface treatment.

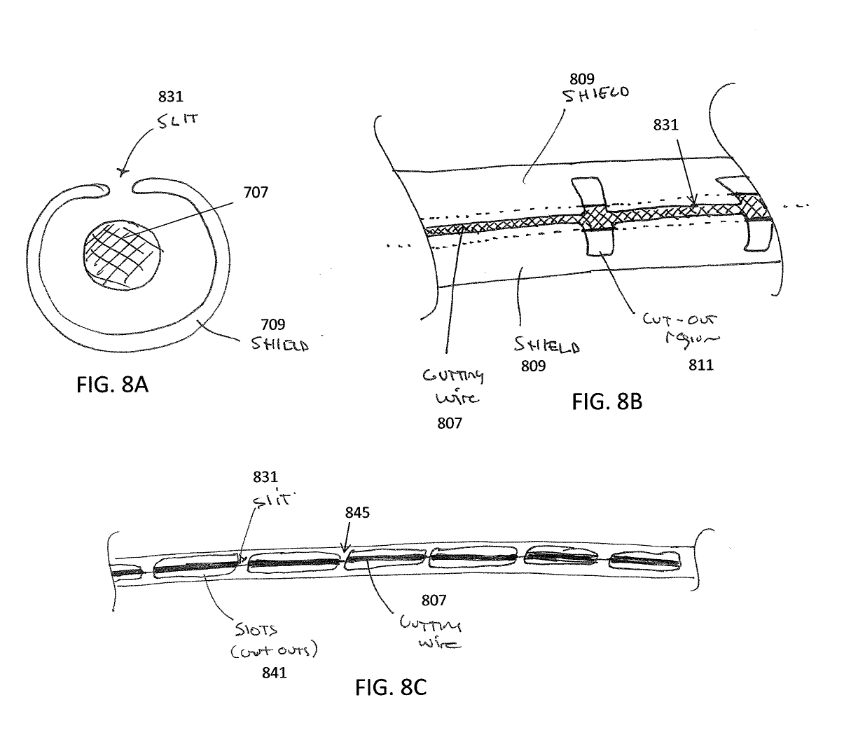

The shield may also include a longitudinal exit or exit region for the cutting wire, to allow the cutting wire to extend out of the shield and into the target to so that it may cut the target tissue. In FIG. 7B, the exit region is a slit 731 that extends along the distal-to-proximal length of the shield. The slit may be an opening (including a closed opening). The opening may be opened to a width that is less than the diameter of the cutting wire. For Example, FIG. 8 shows one variation of a cross-section through a device such as the one shown in FIG. 7B, including a cutting wire, shield 709, and slit through the shield 731. Other cutting wire exits regions may include a longitudinal gap or opening, a perforated region that is configured to be opened by the cutting wire so that the cutting wire may exit the shield during use, or the like.

In some variations, the shield concentrically surrounds the cutting wire when the device is in the resting state, as shown in FIGS. 7A and 7B. In this example, the shield extends from a proximal shaft 721 over the cutting wire 707 and is fixed (along with the cutting wire) to the distal guidewire coupler 705. A biasing or elastic element 715 is attached to the proximal end of the shield and connects the shield to the proximal end of the device. In FIGS. 7A and 7B, the bias is a spring element that allows the proximal end of the shield to move (including extend distally) relative to the rest of the device (including the cutting element) from the proximal end, while applying a restoring force to return the shield to the neutral position shown in FIG. 7A. Thus, the bias 715 will allow the shield to extend along a longer path from the proximal to distal ends than the cutting wire in operation, while keeping the shield in tension to pull it towards the cutting wire (and away from the non-target tissue direction).

As shown in FIG. 7A in a side view and FIG. 7B in a top view, the bias (e.g., spring) is positioned at the proximal end, near the handle. In some variations, the bias is at the distal end (e.g., near the guidewire coupler).

In some variations, the bias is not a spring, but is a region of the shield that has elastomeric properties. For example, the proximal or distal ends of the shield may be an elastomeric material (or coupled to an elastomeric material), such as a rubber material. In some variations the entire shield is formed of an elastomeric material (or has elastomeric properties).

FIGS. 7C-7J illustrate an alternative example of devices, including a handle 703 that can be used in both single-wire cutting devices and double-wire cutting devices, or with cutting devices with any number of cutting wires 707. For example in FIG. 7C, the handle 703 can have a shaft 721 (in this example, an elongate, rigid member) on which a biasing or tensioning element 715 is disposed. In some examples, the bias can be a spring. The shaft 721 can have a distal stop 722 that limits the distal travel of the bias 715. In some examples, the distal end of the bias 715 can be attached to a lock. In FIGS. 7C-7J, the lock includes a ring 716 that is configured to slide over the shaft 721. In some variations the lock may be a crimp, tab, or other surface that engages with one or more, preferably one-way, locking regions on the shaft. In general, the lock acts to prevent the bias from retracting (and thereby retracting any backing/shield/spacer to which it is attached) proximally, e.g., towards the handle. In FIGS. 7C and 7D, the distance between the ring 716 or distal end of the bias 715 in the unstretched state and the distal stop 722 is the available stretch, which is the difference in length of the bias 715 between the unstretched state and the fully stretched or fully retracted state. In order to increase the available stretch, and thereby allow the cutting device to cut facets or other bones having a larger diameter, the distance between the distal stop 722 and the ring 716 or distal end of the bias 715 can be increased (e.g., by elongating the shaft). In some examples, the available stretch is increased by moving and positioning the bias 715 more proximally, such as into the handle 703, such that the distal end of the bias and the ring 716 are located more proximally in the unstretched state. This is illustrated in FIG. 7C. In some examples, the available stretch can be increased to about 60 mm or more, which can allow the cutting device to treat a 35 mm diameter facet joint. In FIGS. 7C and 7D, only the proximal region of the device is shown; one or more cutting wires as described above may extend from the distal end of this region and may include a guidewire coupler at the distal end (not shown).

In some examples, a backing, such as a shield 709, or support, or guide, can be attached or connected to the bias 715 and/or the one-way lock (e.g., ring 716). As the bone is cut, the wires are pulled through the bone and/or tissue, leaving the backing along substantially the same path taken by the device initially. In this example, as the bone is cut the bias 715 may move to a fully retracted or fully stretched position; in this position the bias 715 can exert a significant proximally directed force or tension to the backing (shield) which may cause the shield 709 to eject the cut bone segment out of the body at the completion of cutting. To reduce or prevent this ejection of the cut bone segment, the device may include a bias 715 of reduced stiffness that generates less spring force. For example, the spring force may be between about 0.1 lb/in and about 5 lb/in, between about 0.2 lb/in and about 3 lb/in, between about 0.5 lb/in and about 2 lb/in, or in some variations, less than about 3 lb/in, less than about 2 lb/in, less than about 1 lb/in, less than about 0.5 lb/in, less than about 0.3 lb/in, less than about 0.2 lb/in, or less than about 0.1 lb/in. In some variations, the spring constant of the bias is about 0.47 lb/in. In addition, the shaft 721 can be provided with one or more retention tabs 723 (e.g., one-way lock positions) that allow the ring 716 to travel distally over the tabs 723 in one direction, e.g., distally, but prevent or inhibit the proximal travel of the ring 716 back over the tabs 723. For example, the lock positions may be tabs 723 such as strips or segments of the shaft that are biased radially outwards at the distal ends of the strips or segments. The tabs 723 can be located at strategic locations on the shaft 721 to reduce the amount of return bias 715 travels after fully cutting through the bone. As the bias 715 stretches during use, the ring 716 will pass over the tabs 723 and then lock distal to the tab 723, thereby limiting the amount of return travel and reducing the amount of spring force on the bone fragment. For example, the tabs 723 can be located at ⅓ or ¼ intervals along the shaft 721. In some examples, the tabs 723 can be located at the ⅓ and ⅔ positions along the shaft 721.

FIGS. 7E to 7J illustrate the extension of the bias that allows the backing to remain behind in the tissue (e.g., along the initial path taken by the device) when the cutting wire(s) are cutting into the tissue. These figures also illustrate the use of a lock as described above, limiting the force applied by the backing against the tissue being cut. For example, in FIG. 7E, the device is configured as an elongate, bimanually controlled tissue modification device for cutting tissue in a patient that includes a proximal handle (not shown), an elongate shaft 721, a pair of adjacent cutting wires 753, 753′ extending from the distal end of the shaft, and a backing (in this example configured as a spacer 755) that extends alongside the shaft and behind the cutting wires to hold them spaced apart, and connects to the distal end region of the device (not shown). The proximal end of the backing is connected to the distal end region of the bias 715, which in this example is a spacer bias that is a spring element including a lock configured as a ring 716 that can slide along the elongate shaft 721. The bias is housed at least in part, within the handle. FIG. 7F shows an enlarged view (view F) of FIG. 7E and these figures illustrate the device before cutting tissue, when the spacer 755 holds the cutting wires a predetermine distance apart and moved with the cutting wires as the cutting region of the device bends, for example, during positioning around a target tissue.

FIGS. 7G and 7H illustrate the device of FIGS. 7E and 7F during operation, when the cutting wires are cutting into tissue such as bone (not shown). When cutting, the cutting wires advance up (in the orientation of this figure) as the device is pulled and reciprocated so that the cutting wire cut into the tissue, after disengaging from the spacer 755. As the cutting wires are pulled through the tissue, the spacer is left behind, and may reciprocate with the device in substantially the same path that the device was initially placed, when positioning around the target tissue. In contrast the path of the adjacent (e.g., parallel) cutting wires may extend up through the tissue as it is being cut. As shown in FIG. 7G, this results in the cutting wires 753, 753′ separating from the backing (spacer 755). The bias 715 extends distally, allowing the backing (spacer 755) to remain in essentially the same path around the tissue being by effectively extending the length of the backing and bias. FIG. 7H shows an enlarged region (H) of FIG. 7G.

FIGS. 7I and 7J illustrate the operation of the device of FIGS. 7E-7H with the backing (shield 755) fully extended by the bias 715. In this example, the bias is prevented by the lock (ring 723) engaging with the locking region (tab 723 on the shaft), limiting the force that the backing member can apply on the tissue and preventing the backing from retracting and ejecting the cut tissue from the patient.

FIG. 8A shows a section thorough a region of the shielded cutting wire near the middle of the device, showing the shield concentrically surrounding (or partially surrounding) the cutting wire. None of the figures shown herein are to scale, and in this instance the shield may be separated from the cutting wire or it may be immediately surrounding the cutting wire (e.g., the inner diameter of the shield may be approximately the same as the outer diameter of the cutting wire. The slit 831 at the top of the section is an opening that is smaller than the outer diameter of the cutting wire 707. In some variations the cutting wire is completely encased in the shield. The shield may have a uniform thickness, or it may have an unequal thickness. For example, the shield may be thicker on the bottom region of the device. In general, in some variations the devices described herein are oriented so that they have an upper (e.g., cutting) direction and a lower (e.g., shielded) direction. The devices may be marked or otherwise labeled to indicate the orientation. For example, the devices may be configured to orient so that they bend more readily in a particular direction or plane. In some variations the device have a flattened, ribbon-like length (e.g., with a rectangular or oval-shaped cross-section). The handle and/or guidewire coupler may be configured so indicate the direction of the more middle regions of the device.

FIG. 8B shows a portion of a middle region of a device similar to the variation shown in FIG. 7B. In this example, the cutting wire 807 is surrounded by the shield 809, except for cut-out regions 811 and the longitudinal slit 831.

Another variation of a shield is shown in the top view of a device shown in FIGS. 8C. In this example the shield includes a plurality of cut-out regions 841 that extend along the length of the device and provide flexibility, and may also make it easier for the cutting wire to engage with the target tissue, and exit the shield to cut into the tissue. The cut-out regions 841 may be configured as slots or windows, and they may extend circumferentially only partially around the shield (e.g., facing the upper or tissue engaging side of the device, so that the shield remains solid on the lower (non-target tissue facing) side. In this example, the windows (slots 841) on the upper surface may be separated by shield regions 845 that may include slots and/or perforations from which the cutting wire 807 may exit when cutting tissue. In some variations this upper region of the shield does not include a separate slit or opening, but the shield is configured (with the large window regions) such that the cutting wire may cut through these relatively small regions of shield separating the openings 841. These regions may also be prepared for cutting by the cutting wire, e.g., by having a pre-formed trench, channel, or the like (similar to the perforations mentioned above).

In any variation of a shielded single-wire tissue modification device, the device may be configured so it couples to a guidewire. A guidewire coupler may be attached to the distal end of the device; in some variations the cutting wire may be secured to a guidewire coupler. For example, FIGS. 9A-9C illustrate one variation of a device including a guidewire coupler region at the distal end of the device. In FIG. 9A the shield region is transparent, revealing an inner cutting wire 907 surrounded by the shield region 909. FIG. 9B shows a portion of the distal end of the device, including an attachment region for the shield 933 and a guidewire coupler 955 at the distal end. FIG. 9C shows an enlarged portion of the distal end region (box C), including the attachment to the shield, the attachment of the cutting wire to the guidewire coupler, and the guidewire coupler.

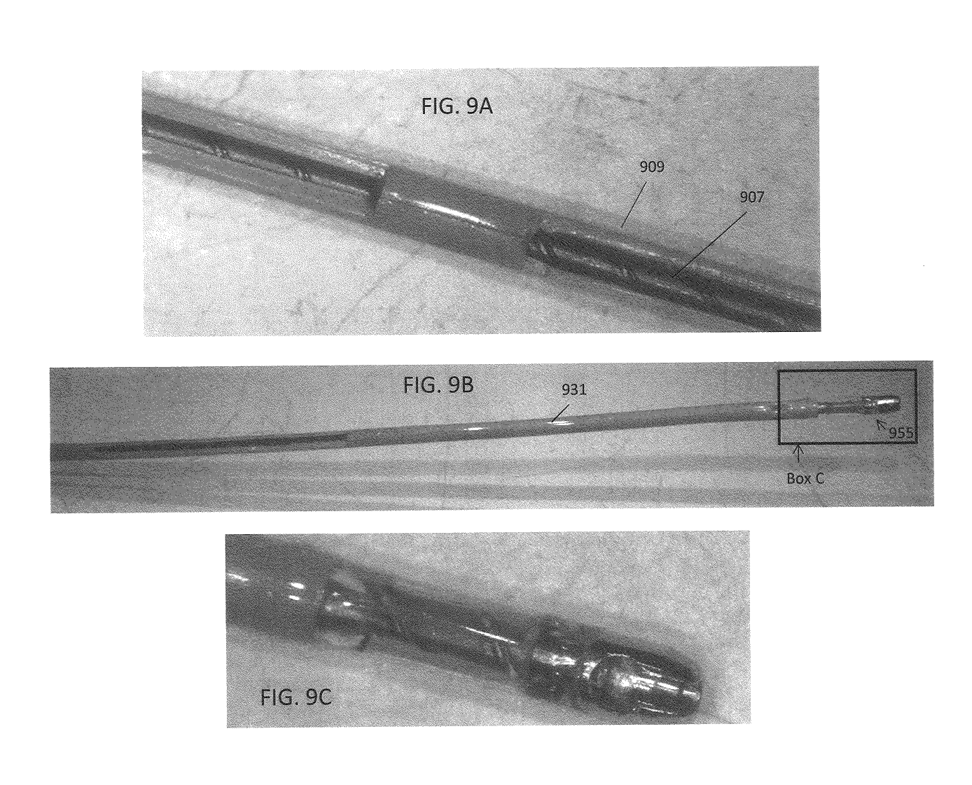

FIGS. 10A-10C illustrate a portion of the proximal portions of a device similar to that shown in FIGS. 7A and 7B. In this example, the proximal handle 1011 is connected to a shaft region 1021 that may be rigidly or flexibly connected to the more distal regions (including the cutting wire). A bias (spring 1015) is secured to the shield 1009, as described above, allowing the shield to extend and/or retract longitudinally (e.g., proximally to distally) during use.

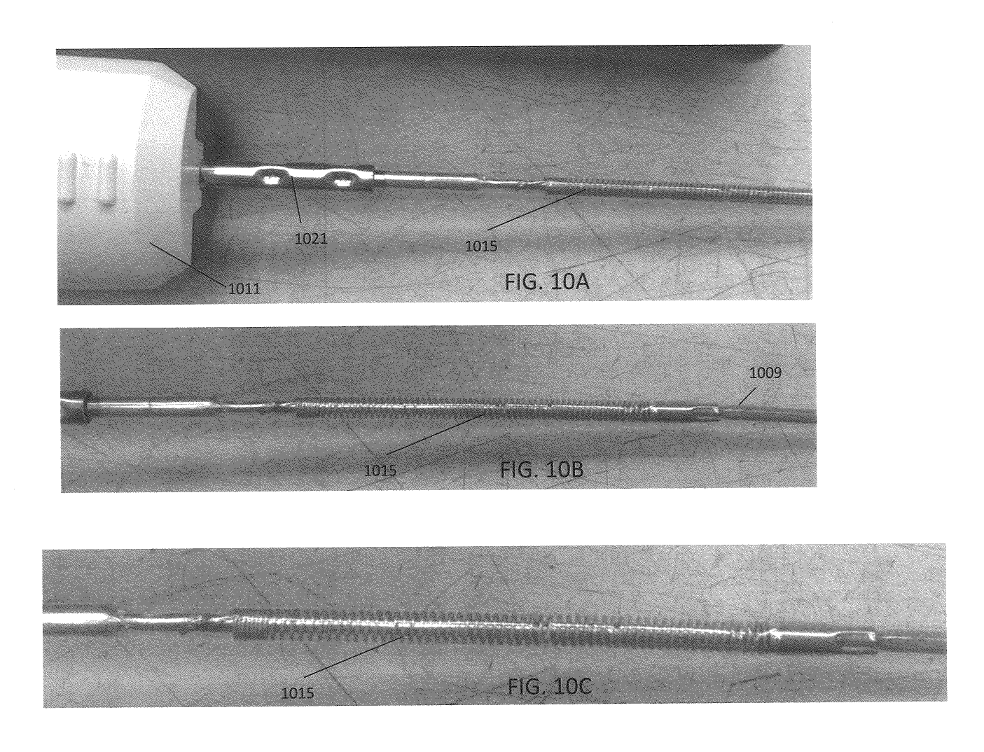

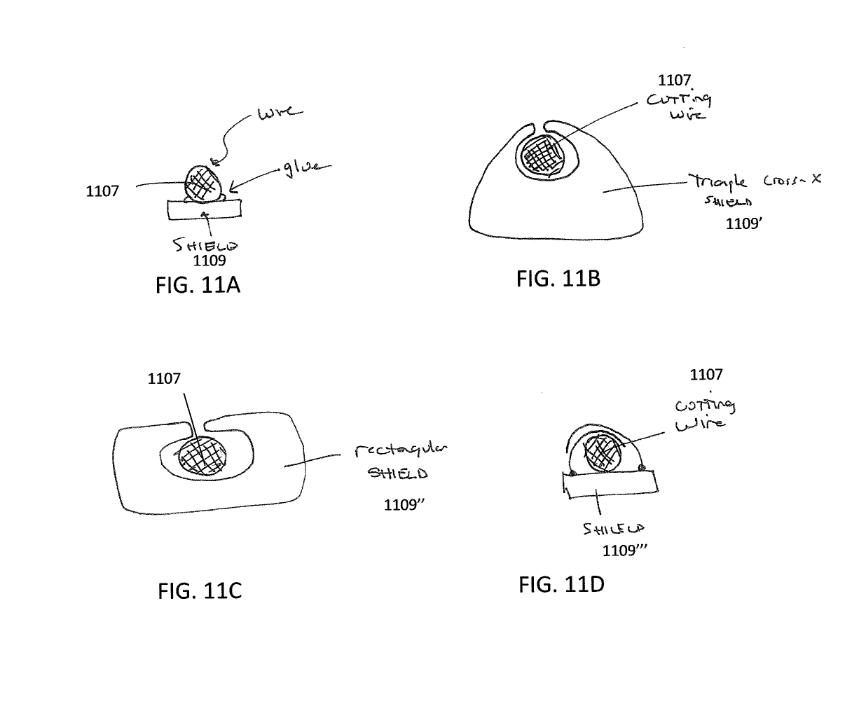

Alternative shield examples are illustrated in FIGS. 11A to 11D. For example, as mentioned above, the shield may be placed only on one side or circumferential portion of the cutting wire, as shown in FIG. 11A. In this example, the shield is positioned on the bottom surface, and the cutting wire 1107 is releasably secured (e.g., using a frangible glue or adhesive) to the shield. The shield 1109 is an element that may be separated from the shield during use. The shield as a larger diameter than the cutting wire in the region of the device configured to cut the tissue, thus, this region may be left behind after breaking from the cutting wire when the device cuts into the tissue. Other portions of the shield may be configured differently (e.g., proximal and distal regions may circumferentially enclose the cutting wire. Thus, the shield may have different cross-sectional profiles along the length of the device.

FIG. 11B shows another variation of a shield 1109′ in which the cutting wire 1107 is at least partially surrounded by a shield element 1109″ having a widened/enlarged base region; this configuration may also provide a greater thickness than the upper region including the slit through which the cutting wire may exit. Similarly, FIGS. 11C and 11D show other variations in which the cutting wire 1107 is protected by a shield 1109″, 1109′″. The shield shown in the cross-section of FIG. 11C has a rectangular cross-section. In FIG. 11D the shield 1109′″ is formed (at least in the illustrated section) as overlapping leaves of material on the upper surface.

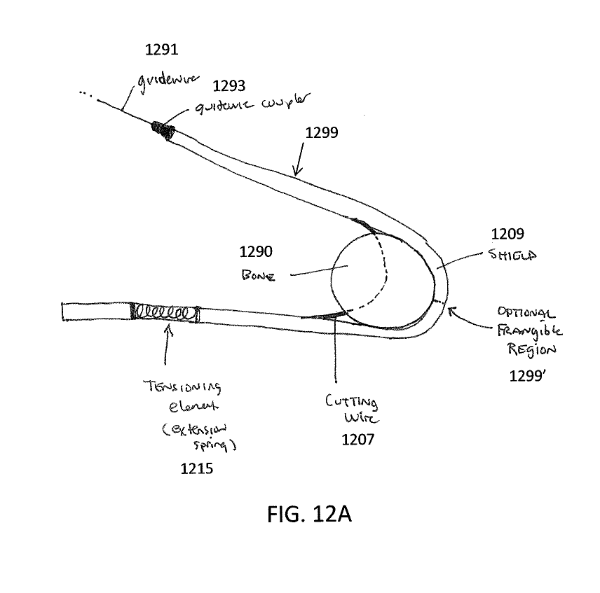

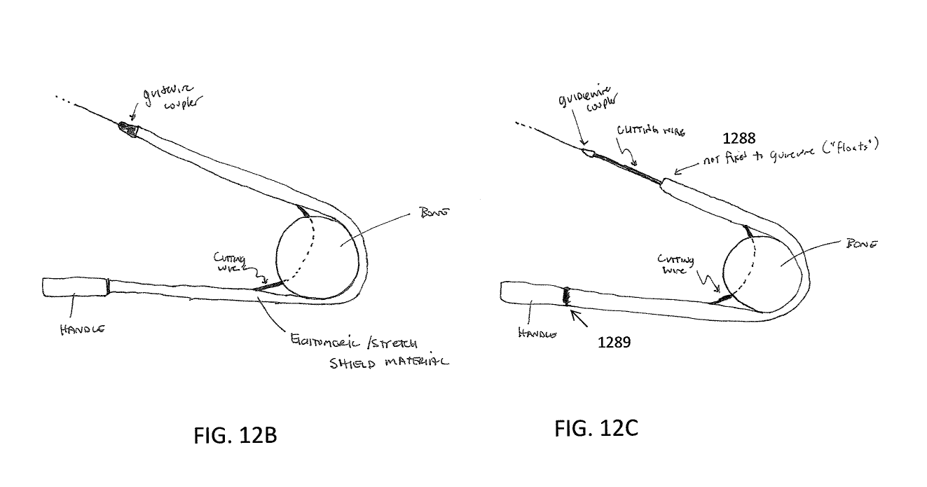

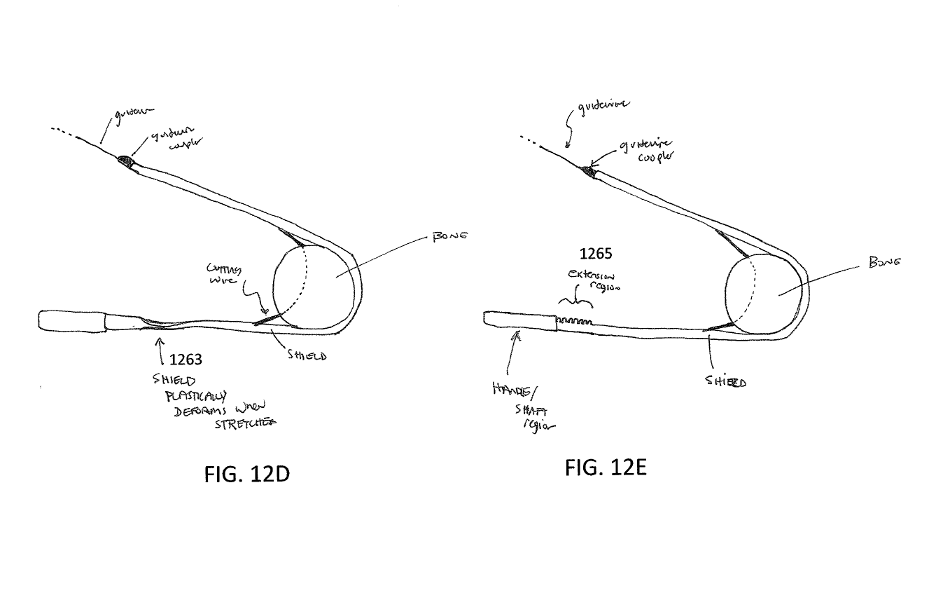

FIGS. 12A-12E illustrate operation of different variations of shielded single-wire tissue modification devices. In FIG. 12A the shielded single-wire tissue modification device is similar to that shown in FIGS. 7A and 7B, and is shown in the middle of cutting a bone 1290. The device may be pulled into position as discussed above, by placing the guidewire around the target tissue to be cut (e.g., threading a guidewire around the target tissue using a cannula, probe, etc.) then coupling the proximal end of the guidewire (which may include an enlarged region, hook, hitch, etc.) to the distal end of a shielded single-wire tissue modification device. The shielded single-wire tissue modification device can then be pulled into position, and reciprocated by applying tension from both ends and reciprocating the device. The guidewire 1291 may remain coupled to the shielded single-wire tissue modification device and a handle attached to the guidewire; alternatively a handle may be directly attached to the shielded single-wire tissue modification device at the distal end. While cutting, the shield 1209 remains outside of the cut tissue, while the cutting wire 1207. Since the shield 1209 in this example is attached to the distal end (e.g., to or near the guidewire coupler 1293) this the shield may extend as the cutting wire is pulled through the tissue, by extending the tensioning element/region 1215. In this example the tensioning element is a biasing spring element. Once the cutting wire has passed through the target tissue (e.g., bone 1290), completely or partially, the device may be removed from the tissue. In some variations, it may be desirable to cut either the shield and/or the cutting wire so that the two elements (which may now extend through different paths through the tissue) may be removed without damaging non-target tissues. For example, the device may be cut proximally or distally (e.g., see arrow 1299). And both ends removed separately by pulling distally and proximally. Alternately, in some variations a region of the shield may be made frangible or breakable so that it can be intentionally separated after cutting through the tissue.

FIG. 12B shows another variation of a method of using a shielded single-wire tissue modification device. In FIG. 12B the shielded single-wire tissue modification device includes an elastomeric shield. The elastomeric shield may stretch to extend as the wire cuts though the tissue, while remaining behind in the tissue.

The shields of the shielded single-wire tissue modification devices shown in FIGS. 12A and 12B are fixed at both their proximal and distal ends, and thus may reciprocate with the cutting wire as the devices are operated. Both shields may be held in tension against the target tissue being cut (bone) because of the tensioning elements, the elastomeric shield in FIG. 12B and the bias 1215 in FIG. 12A. However in some variations, one or more ends of the shield may be released or releasable from the movement of the device when operating the shielded single-wire tissue modification device. For example, in FIG. 12C, the shield may “float” at one or both ends, so that the cutting wire may be reciprocated while the shield remains in position within the tissue. In FIG. 12C, the distal end of the shield 1255 is uncoupled from the distal end of the device (e.g., the guidewire coupler). In some variations the proximal end of the shield 1289, may also be uncoupled from the rest of the device. In both variations the shield may be uncoupled from either or both end after the device has been positioned within the tissue. For example, the device may be positioned using a guidewire as discussed above, and, with both ends of the shield external to the patient, one or both ends uncoupled. Thus, the shield may be connected at one or both ends with a removable coupler (including a threaded, snap, or other coupler. In some variations the shield may be cut to remove it from the cutting wire so that it can remain in position as the rest of the device is reciprocated to cut target tissue.

Alternative variation of shields for shielded single-wire tissue modification devices are shown in operation in FIGS. 12D and 12E. In FIG. 12D the shield includes a plastically deformable region 1263 (located proximally in this example) that deforms when tension is applied, stretching to allow the shield to remain outside of the tissue as the cutting wire cuts through the bone, as shown. In FIG. 12E, the shield includes an extendable region 1265 that may controllable extend as the cutting wire cuts through the tissue. The extendable region may include a ratcheting or zip-tie like release mechanism for releasing increments of shield.

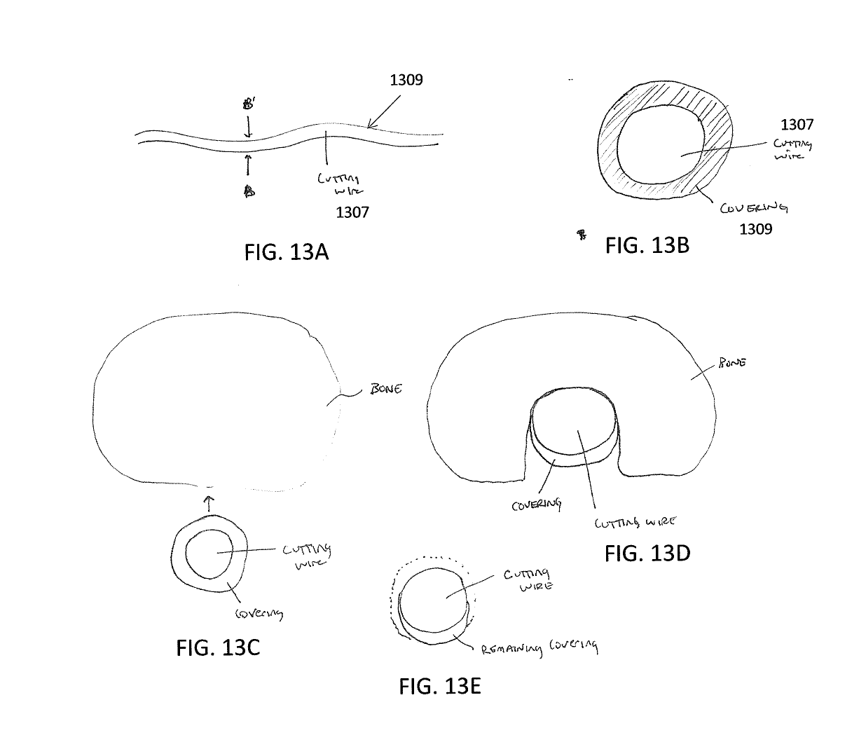

Another variation of a shielded single-wire tissue modification device is shown in FIGS. 13A-13E. In this variation, the shielded single-wire tissue modification device includes a cutting wire 1307 that is coated with a shielding material that can be worn off during reciprocation on the cutting side of the device, as illustrated in FIGS. 13C and 13D. A cross-section through the device is shown in FIG. 13A. The cutting wire 1307 is coated with a shielding covering material 1309. The coating material may be a wearable material, such as a bone wax (e.g., paraffin), a silicone material, or the like. Moving the shielded single-wire tissue modification device including the wearable coating against the target tissue may wear away the protective coating material, exposing the cutting wire and cutting the target tissue. This is illustrated in FIG. 13E, in which the dashed lines indicate the region where the coating has been worn away, as also shown in FIG. 13D. In some variations the coating may also enhance the quality of the cut, for example, when the coating is a bone wax that may reduce bleeding and enhance healing.

### Adapters for Forming Dual-Wire Tissue Modification Devices

Any of the shielded single-wire tissue modification devices described herein may also be used to form a two-wire tissue modification device. For example, a pair of shielded single-wire tissue modification device may be coupled to an adapter before or after being pulled into position in the tissue around the target tissue. For example, a dual-wire probe, such as those described in U.S. patent application Ser. No. 13/757,661, previously incorporated by reference in its entirety, may be used to pull a pair of guidewires into position so that both shielded single-wire tissue modification devices can be pulled in to position.

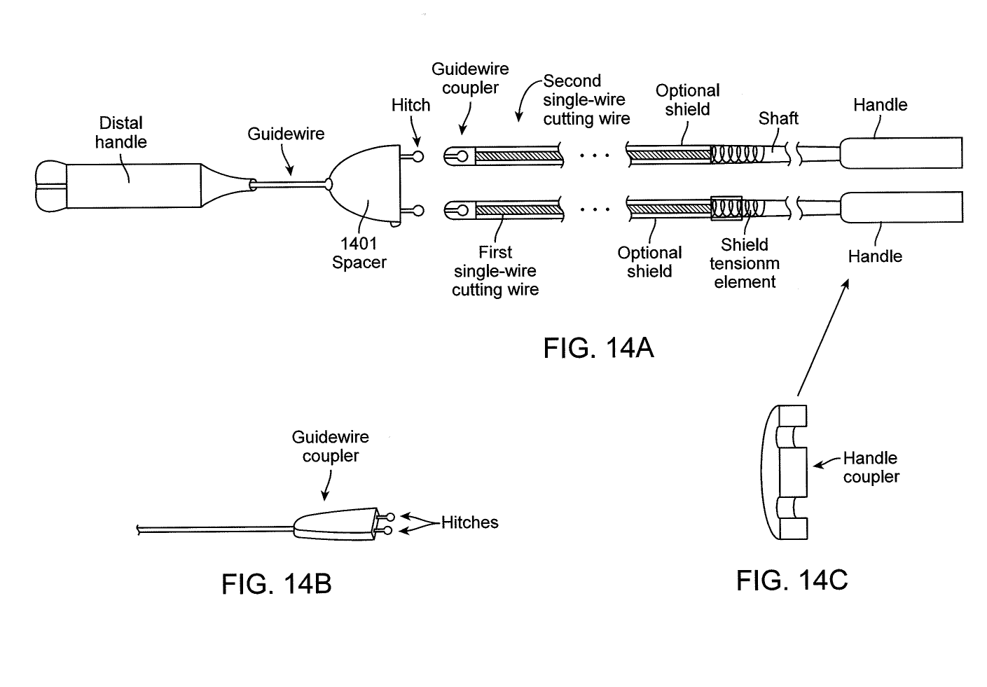

For example FIG. 14A shows one example of an adapter that may be used with two shielded single-wire tissue modification devices to form a pair of cuts into the tissue. The distal handle may couple to the adapter or spacer 1401. The spacer/adapter 1041 is shown in a side view in FIG. 14B, and includes two coupling regions configured to have the same geometry as the end of the guidewire that may be held by the guidewire couplers on the shielded single-wire tissue modification devices. In FIGS. 14A and 14B, this configuration includes an enlarged region (“hitch”) that may securely mate with the guidewire coupler. The spacer/adapter 1401 may hold the shielded single-wire tissue modification devices a fixed distance apart. In some variations a separate handle coupler (shown in FIG. 14C) may also be used to couple the proximal handles of the shielded single-wire tissue modification devices together at the same fixed distance apart. In some variations the handles themselves are adapted so that they can lock together at the desired separation.

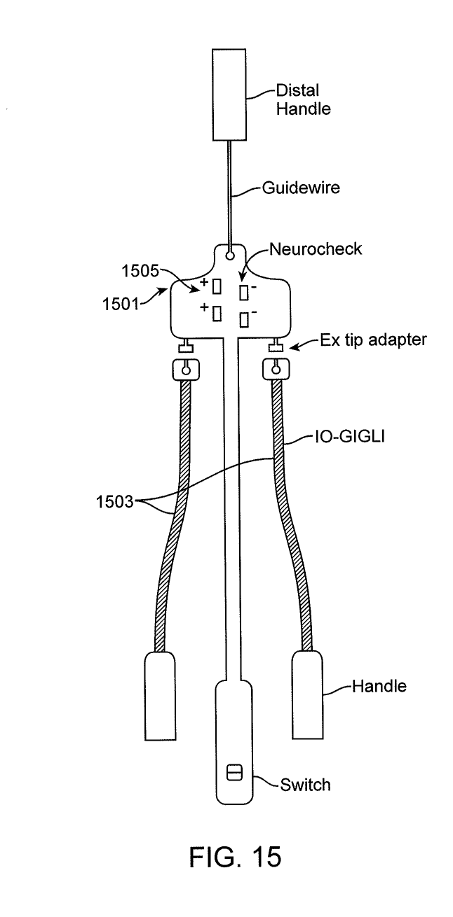

Any of the methods and devices described herein may also be configured to check and/or confirm the position of nearby neural tissue. A separate neural check device may be used to confirm that a nerve is not in the direction of the target tissue to be cut from the path taken by the guidewire. Alternatively, the devices may be configured to include an integrated electrical stimulation system. For example, an adapter/spacer may include one or more sets of electrodes as part of the adapter/spacer that connect to two or more shielded single-wire tissue modification devices. The electrodes may emit energy in a first and/or second direction (e.g., upper/lower directions) to determine if a nerve is in the direction to be taken by the cutting wire prior to cutting. FIG. 15 shows one example of this, in a system including a spacer 1501 to which a pair of shielded single-wire tissue modification devices 1503 may be attached. The spacer includes a set of electrodes 1505 on each side that can be connected to a power supply and used to directionally emit energy to confirm a nerve is not in the path of the cutting element prior to cutting.

Facetectomy Devices with Cutting Rungs

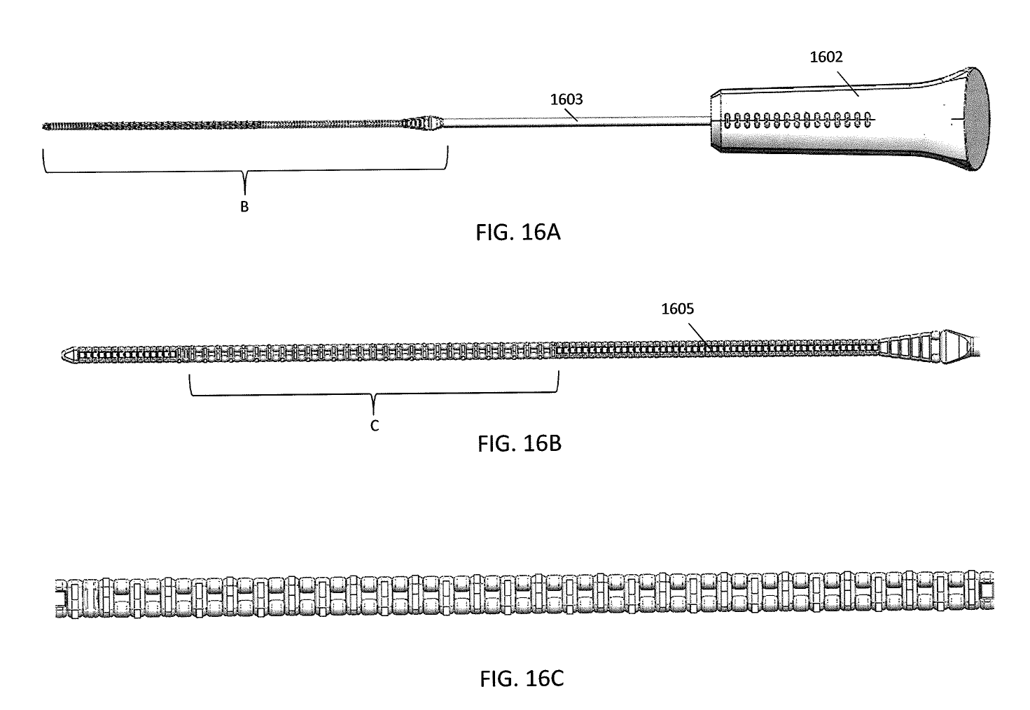

FIGS. 16A-18D also illustrate cutting devices having cutting blades configured as cutting teeth that extend from a cutting surface in a tapered manner, so that the base of a cutting tooth is smaller than the region opposite of the base (the “top”). This may allow the cutting teeth to extend over the base region and permit aggressive cutting that is not limited to a cutting depth based on the height of the cutting surfaces (e.g., the height of a cutting blade projecting from the base). The devices described herein are well suited to use as part of a tissue modification device having cutting blades (e.g., “teeth”) that extend upwards from a flexible base, particularly device having rungs (cutting rungs), however these cutting features may be incorporated into any of the variations described herein, including single-wire devices, or devices having multiple parallel wires. Further, any of the features described herein, including features of the single-wire and shielded devices and methods of using them, may be incorporated as part of these devices.

In particular, the devices having the box-shaped, tapered based cutting blades described herein may be particularly useful for performing a facetectomy, and my therefore in some variations be referred to as a facetectomy device or apparatus.

For example, a rapid facetectomy device may be formed using a plurality of cutting rungs that include the box-shaped, tapered base cutting blades. The device may have modular construction including a plurality of rungs forming an upper surface (“base”) that is opposite a lower surface. The blades may extend from the upper surface of the cutting rungs, while the lower surface is smooth and atraumatic, preventing damage to tissue as the device is reciprocated to cut.

The proposed devices including the box-shaped, tapered base cutting blades may have a greater cutting efficiency than other devices with blades that are pointed, rounded or curved, because such blade designs do not typically cut wider than the base of the device. Thus, there may be sections of bone (e.g., at the outer most edges of such devices) that are not cut by the blades; this may limit the depth of the cut, limiting the cutting depth into bone.

Devices having the tapered or canted blades described herein may arrange the blades so that they extend beyond the width of the base of the device (e.g., the upper surface of the cutting region of the device), thereby allowing the blades to cut a path through the bone which is wider than the base section of the device. To vary the aggressiveness of the blades, the overall height of the blades and the canted angle of the blade can be adjusted.

Such devices may have an advantage over other single wire, double wire, or ribbon-shaped devices (elongated, flat devices) because in some variations a bottom shield is not necessary. In particular, the bottom of the device may be smooth and safe to soft neural tissues.

In operation, such devices may function as described above for the single-wire or multiple-wire devices. For example, a guide wire may be placed, attached to the distal end of the device, and then pulled into a foramen with a distal handle. The device may then be reciprocated until the device fully cuts through the tissue (e.g., facet joint).

FIGS. 16A-16C illustrate one variation of a device including tapered or canted blades described herein. In this variation, the canting outwards of the blades enables a self-clearing by the device for ongoing depth of cut, as described below.

For example, in FIG. 16A, the device includes a proximal handle, a rigid shaft 1603 extending distally from the proximal handle 1602, and a flexible distal region extending from the distal end of the rigid shaft, which includes a guide wire coupler at the distal end. The proximal rigid shaft may be optional, as the entire device may be flexible, and/or may coupled directly to a proximal handle. The flexible distal end of the device, region B, of the device is shown in more detail in FIG. 16B. In this variation, the distal end is flexible in one plane (e.g., up/down relative to the plane of the paper, but is relatively rigid with respect to the side-to-side motion; this is because the device is formed of a plurality of interconnected rungs 1605. The more proximal rungs are atraumatic, while the more distal rungs are configured as cutting rungs, including cutting elements projecting up from the top surface of the rungs. The rungs may be connected, for example, by one, two, or more cables. The rungs may be directly connected, or may be separated from adjacent rungs by a spacer. This is shown in more detail in FIG. 16C, which illustrates the cutting region, region C.

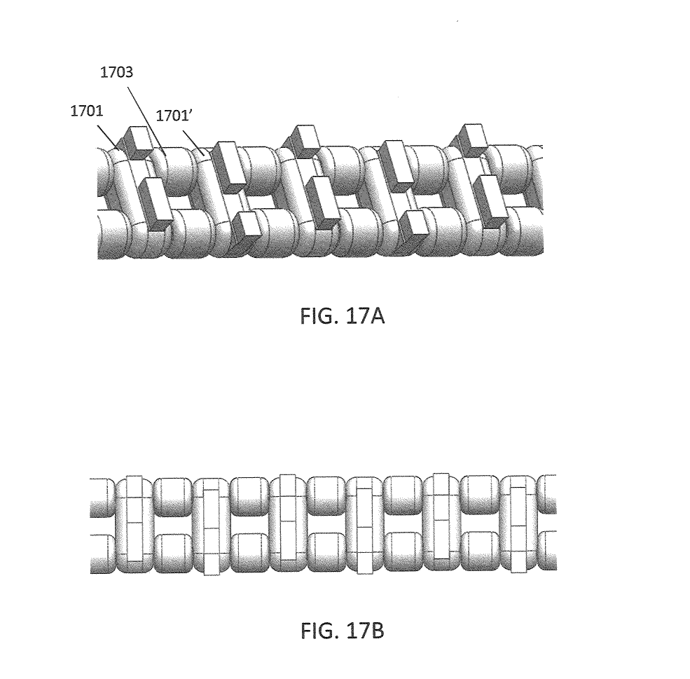

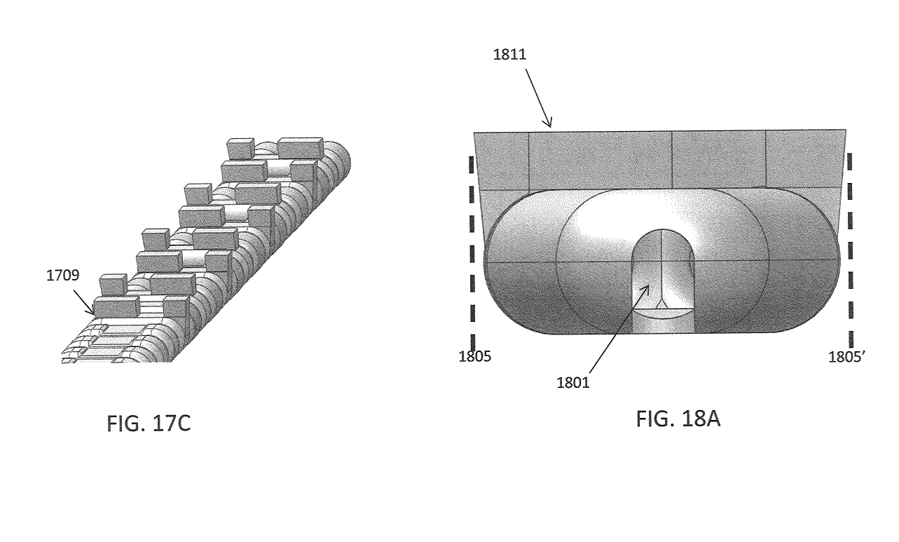

FIG. 17A shows a perspective view of the upper surface (cutting surface) of the cutting region (region C) of the device of FIG. 16A. In this example, cutting rungs 1701, 1701′ are separated by separators 1703. A space (through which bone or other debris cut from the tissue may pass, is formed between adjacent rungs. The separators may be rounded or have curved shoulders to help with flexibility.

In this example the cutting rungs include tapered box-shaped blade elements (“teeth”) that extend up from the top surface of each rung. Two such cutting blade elements are shown on each cutting rung, with a space between them. The location of the spaces is offset relative to adjacent cutting rungs, which may help in balancing the cutting. For example, the same cutting rung shape may be flipped to provide balanced coverage (preventing the device from sliding when reciprocating in the tissue). This space (slot) within the blade of the cutting rung may allow for passage of bone and tissue to minimize clogging of the device in operation.

In contrast to the cutting surface shown in FIG. 17A, showing the upper/top surface or plane of the device, FIG. 17B shows the bottom surface of the device, which is atraumatic, e.g., smooth.

FIG. 17C show a front perspective view of the apparatus, including the cutting teeth projecting up from the base formed by the upper surface 1709 of the cutting rungs. As will be shown in more detail below, each of these cutting elements is tapered slightly from the top of the box-shaped cutting element (furthest from the cutting rung) to the base of the cutting element (adjacent to the cutting rung). Thus, the top of the device, furthest from the rung, has a larger surface area than the bottom (attached to the rung). Not that “attached” and bottom are relative; the rung may be formed with the cutting element; in this case the cutting element or blade is integral with the rest of the rung, and base or bottom refers to where the box-like projection extends from the portion of the rung that is on the same level/height as the adjacent spacer and/or non-cutting rungs.

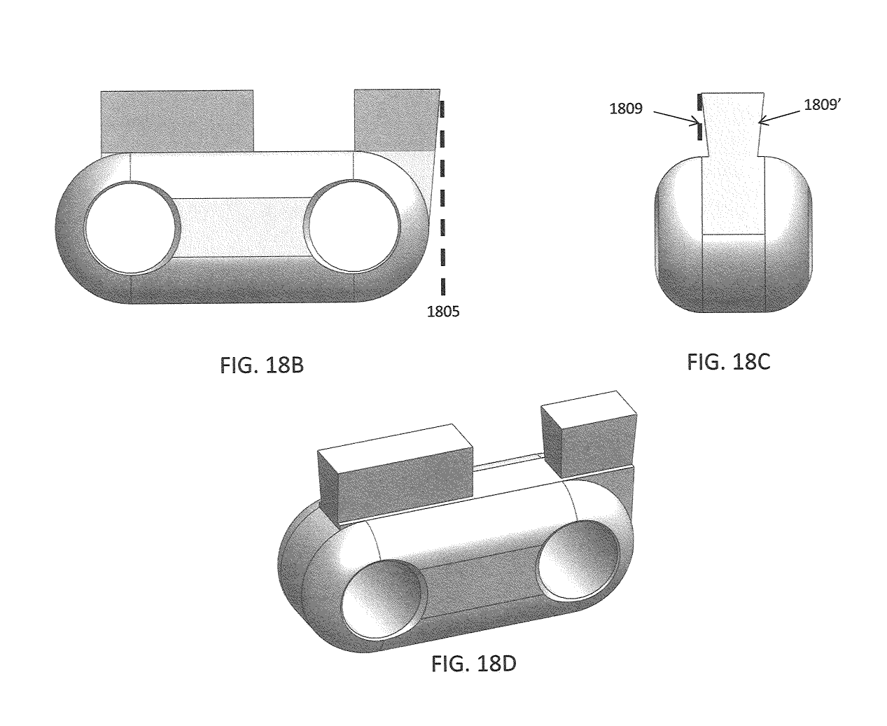

FIG. 18A is an end view of the distal end of the device including the cutting region. The guidewire coupler 1805 is illustrated. The rungs are tapered or canted, as shown, extending from the base of the rung at an angle on both sides of the device; the dashed lines 1805, 1805′ are included to highlight the angle. In general, the angle may be between 2° and 45°, typically between 3° and 15°, between 3° and 10°, etc. In FIG. 18A, the angle is approximately 5°, relative to the vertical (assuming that the top of the cutting element 1811 is horizontal, as is the upper rung surface). Any of the four generally vertical sides of the box-shaped cutting element may be canted, as illustrated below. In some variations only one or both of the “outer” vertical surfaces are canted, while in other variations the vertical surfaces are canted.

FIG. 18B shows a single cutting rung, including two cutting teeth, which are configured as tapering box-shaped elements extending from the upper surface of the rung. In FIG. 18B, the cutting tooth on the right extends over the outer edge of the rung, so that the top (surface furthest from the upper surface of the rung) extends laterally further than the outer width (lateral) of the rung, as shown by the dashed line 1805. In this case, the lateral walls of the second cutting tooth on the rung are not canted, and neither is the inner lateral wall of the first cutting tooth. Further, the second cutting tooth is positioned laterally inward from the edge of the cutting rung. However, as shown in FIG. 18C, both the medial surfaces 1809, 1809′ of the box-shaped cutting element are canted so that the device tapers. The angle of taper on the lateral and medial surfaces may be the same or different, but may generally be between 2° and 45° (e.g., between 2° and 30°, between 2° and 25°, between 3° and 20°, etc.). The aggressiveness of the cutting by the blade elements may be determined by angle of blade and height of blade. A blade having canted surfaces (tapered) may promote cutting wider than main body of the device. FIG. 18D shows a perspective view of this cutting rung.

As mentioned, none of the figures included herein are necessary to scale, unless the context indicates otherwise. Also as used herein in the specification and claims, including as used in the examples and unless otherwise expressly specified, any numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

Although much of the previous description and accompanying figures generally focuses on surgical procedures in spine, in alternative examples, devices, systems and methods of the present invention may be used in any of a number of other anatomical locations in a patient's body. For example, in some examples, the flexible tissue modification devices, including the shielded wires, of the present invention may be used in minimally invasive procedures in the shoulder, elbow, wrist, hand, hip, knee, foot, ankle, other joints, or other anatomical locations in the body. Similarly, although some examples may be used to remove or otherwise modify ligamentum flavum and/or bone in a spine to treat spinal stenosis, in alternative examples, other tissues may be modified to treat any of a number of other conditions. For example, in various examples, treated tissues may include but are not limited to ligament, tendon, bone, tumor, cyst, cartilage, scar, osteophyte, inflammatory tissue and the like. Non-target tissues may include neural tissue and/or neurovascular tissue in some examples or any of a number of other tissues and/or structures in other examples. Thus, various examples described herein may be used to modify any of a number of different tissues, in any of a number of anatomical locations in the body, to treat any of a number of different conditions.

The examples and illustrations included herein show, by way of illustration and not of limitation, specific examples in which the subject matter may be practiced. Other examples may be utilized and derived there from, such that structural and logical substitutions and changes may be made without departing from the scope of this disclosure. Such examples of the inventive subject matter may be referred to herein individually or collectively by the term “invention” merely for convenience and without intending to voluntarily limit the scope of this application to any single invention or inventive concept, if more than one is in fact disclosed. Thus, although specific examples have been illustrated and described herein, any arrangement calculated to achieve the same purpose may be substituted for the specific examples shown. This disclosure is intended to cover any and all adaptations or variations of various examples. Combinations of the above examples, and other examples not specifically described herein, will be apparent to those of skill in the art upon reviewing the above description.

What is claimed is:

1. A tissue modification device, the device comprising:
a proximal handle;
a shaft extending distally from the handle;
a backing bias disposed on the shaft;
a cutting wire extending distally from the shaft, the cutting wire comprising a cable and a plurality of ferrules disposed at predetermined locations along the cable;
a guidewire coupler at a distal end of the device; and
a backing coupled to the backing bias and extending adjacent to the cutting wire, wherein the backing is configured to separate from the cutting wire when the cutting wire cuts into tissue.

2. The device of claim 1, wherein the cable comprises a plurality of twisted wires.

3. The device of claim 1, wherein the ferrules are hex crimped to the cable.

4. The device of claim 3, wherein the hex crimps are about ⅓ the length of the ferrule.

5. The device of claim 1, wherein the cutting wire comprises a proximal gap that is free of ferrules and a distal gap that is free of ferrules.

6. The device of claim 1, wherein the proximal gap and the distal gap are each about 5 mm to 25 mm in length.

7. The device of claim 1, wherein the ferrules are spaced between about 0.51 mm and 6.35 mm apart.

8. The device of claim 1, wherein the ferrules have a wall thickness of between about 0.051 mm and 0.25 mm.

9. The device of claim 1, wherein a proximal end of the backing bias is located within the handle.

10. The device of claim 1, further comprising a lock slidably disposed with the shaft and attached to a distal end of the backing bias.

11. The device of claim 10, wherein the shaft further comprises one or more retention regions configured to engage the lock to inhibit travel of the lock in a proximal direction.

12. The device of claim 1, wherein the distance between the distal end of the shaft and the distal end of the backing bias is at least about 60 mm.

13. The device of claim 1, further comprising a second cutting wire extending distally from the shaft adjacent to the cutting wire.

14. The device of claim 13, wherein the backing is configured as a spacer that releasably holds the cutting wire and the second cutting wire a distance from one another.

15. The device of claim 1, wherein the backing is configured as a shield.

16. An elongate, bimanually controlled tissue modification device for cutting tissue in a patient, the device comprising:
a pair of flexible, elongate cutting members extending along an elongate length of the device;
a spacer, wherein the spacer is sized and configured to operate in one of two modes:
a first mode, wherein the spacer is coupled to the cutting members such that it holds a portion of each of the two cutting members a distance from one another, and
a second mode, wherein at least a portion of the spacer is moved away from a cutting member to allow the cutting members to cut further into tissue; and
a spacer bias connected to a proximal end of the spacer and a proximal region of the device, wherein the spacer bias is configured to extend the proximal end of the spacer distally as the spacer is moved away from the cutting member.

17. The device of claim 16, further comprising a lock configured to prevent the spacer bias from retracting the proximal end of the spacer proximally once the spacer bias is extended distally past a predetermined region on the device.

18. The device of claim 16, further comprising locks configured to prevent the spacer bias from retracting the proximal end of the spacer proximally once the spacer bias is extended distally past a plurality of predetermined regions on the device.

19. The device of claim 17, wherein the lock comprises a ring coupled to the spacer bias and configured to engage one or more tabs on a proximal shaft of the device.

20. A tissue modification device, the device comprising:
a proximal handle;
a shaft extending distally from the handle;
a pair of cutting wire extending distally and adjacent to each other from the shaft, at least one of the cutting wires comprising a cable and a plurality of ferrules disposed at predetermined locations along the cable;
a guidewire coupler at a distal end of the device; and
a spacer configured to hold the cutting wires a distance from one another and release the cutting wires as the cutting wires cut tissue.

21. The device of claim 20, wherein the cable comprises a plurality of twisted wires.

22. The device of claim 20, wherein the ferrules are hex crimped to the cable.

23. The device of claim 22, wherein the hex crimps are about ⅓ the length of the ferrule.

24. The device of claim 20, wherein the cable comprises a proximal gap that is free of ferrules and a distal gap that is free of ferrules.

25. The device of claim 24, wherein the cable comprises a proximal gap that is free of ferrules and a distal gap that is free of ferrules, further wherein the proximal gap and the distal gap are each about 5 mm to 25 mm in length.

26. The device of claim 20, wherein the ferrules are spaced between about 0.5 mm and 6.5 mm apart.

27. The device of claim 20, wherein the ferrules are spaced between about 1 mm and 3 mm apart.

28. The device of claim 20, wherein the ferrules have a wall thickness of between about 051 mm and 0.25 mm.

29. A single-wire tissue modification device comprising:
an elongate length of cutting wire extending proximally to distally;
a guidewire coupler coupled to the distal end of the cutting wire;
a shield at least partially surrounding the elongate length of the cutting wire;
a exit region extending along a length of the shield, wherein the cutting wire is configured to pass out of the shield though the exit during use; and
a tensioning element coupled to an end of the shield and configured to extend the length of the shield relative to the cutting wire during use.

30. The device of claim 29, wherein the exit region comprises a slit.

31. The device of claim 29, wherein the exit region comprise a perforated region.

32. The device of claim 29, further comprising a plurality of cut-out regions extending along the length of the shield on an upper surface of the shield.

33. The device of claim 29, wherein the tensioning element comprises a spring coupled to the proximal end region of the shield.

34. The device of claim 29, wherein the distal end region of the shield is coupled to the distal end region of the cutting wire.
